(19) United States
(12) Patent Application Publication
(10) Pub. No.: US 2022/0226434 A1
(43) Pub. Date: Jul. 21, 2022

(54) METHODS OF TREATING HYPERLIPIDEMIA CONDITIONS WITH NETRIN-1 COMPOUNDS

(71) Applicant: The Regents of The University of California, Oakland, CA (US)

(72) Inventor: Hua Cai, Los Angeles, CA (US)

(21) Appl. No.: 17/614,949

(22) PCT Filed: May 26, 2020
(86) PCT No.: PCT/US2020/034484

§ 371 Date: Nov. 29, 2021

Related U.S. Application Data
(60) Provisional application No. 62/854,870, filed on May 30, 2019.

(51) Int. Cl. A61K 38/18; A61P 3/06

(52) U.S. Cl. CPC A61K 38/18; A61P 3/06

(57) ABSTRACT

Disclosed herein are netrin 1 compounds and compositions thereof and methods of using thereof to treat, inhibit, or reduce conditions associated with or resulting from hyperlipidemia.

### CROSS-REFERENCE TO RELATED APPLICATIONS

This application claims the benefit of U.S. Patent Application No. 62/854,870, filed May 30, 2019, which is herein incorporated by reference in its entirety.

### REFERENCE TO A SEQUENCE LISTING SUBMITTED VIA EFS-WEB

The content of the ASCII text file of the sequence listing named “20190527_034044_203P1_seq_ST25” which is 12.1 kb in size was created on May 27, 2019, and electronically submitted via EFS-Web herewith the application is incorporated herein by reference in its entirety.

### BACKGROUND OF THE INVENTION

### 1. Field of the Invention

The present invention generally relates to netrin-1 compounds and compositions thereof for treating hyperlipidemia and related diseases and disorders.

### 2. Description of the Related Art

Netrins and their receptors are well known in the art, as exemplified in U.S. Pat. Nos. 5,565,331; 6,096,866; 6,017,714; 6,309,638; 6,670,451; and 8,168,593; and in US20060019896 and US20060025335.

Netrin-1 is a secreted molecule that is largely known to play a defined role in guiding vertebrate commissural axons in neuronal development. See Kennedy et al. (1994) Cell 78:425-35; Serafini et al. (1994) Cell 78:409-24; and Serafini et al. (1996) Cell 87:1001-14. Recent studies have further demonstrated a critical role of netrin-1 in endothelial cell proliferation, migration, and angiogenic signaling, in addition to morphogenesis of epithelial cells. See Park et al. (2004) PNAS USA 101:16210-5; Carmeliet et al. (2005) Nature 436:193-200; Nguyen et al. (2006) PNAS USA 103:6530-5; Wilson et al. (2006) Science 313:640-4; Liu et al. (2004) Curr Biol 14:897-905. At least eight netrin receptors have been characterized in neurons, vascular system, and other cell types in mammals. These include deleted in colorectal cancer (DCC), UNCSA, B, C, D, neogenin, α6β4, and α3β1 integrins. See Tessier-Lavigne et al. (1996) Science 274:1123-33; Huber et al. (2003) Annu Rev Neurosci 26:509-63; Cirulli et al. (2007) Nat Rev Mol Cell Biol 8:296-306; and Yebra et al. (2003) Dev Cell 5:695-707. Netrin-1 binding to DCC mediates attractive outgrowth of axons, as well as positive angiogenic signaling in endothelial cells. In contrast, the UNC5B receptor appears repulsive, mediating cellular effects such as filopodial retraction, particularly in developing capillaries. See Lu et al. (2004) Nature 432:179-86; and Larrivee et al. (2007) Genes Dev 21:2433-47.

### SUMMARY OF THE INVENTION

In some embodiments, the present invention provides methods of treating, reducing, or inhibiting a hyperlipidemia condition in a subject which methods comprise administering to the subject a therapeutically effective amount of one or more netrin-1 compounds. In some embodiments, the netrin-1 compound is a peptide that has an amino acid sequence that comprises, consists essentially of, or consists of SEQ ID NO: 1 as follows:

| | |
|---|---|
| X1-X2-X3-C-X4-X5-X6-X7-T-X8-G | (SEQ ID NO: 1) |

wherein

X1 is Ala, Asn, Cys, D-Cys, Ser, or Thr, preferably X1 is Cys, D-Cys, Ser, or Thr, and wherein X1 may be linked to the cysteine at the fourth amino acid position or an ethylene oxide compound;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present; and

wherein one or both the amino acid residues at the 10th and 11th amino acid positions may be D-amino acids. In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG. In some embodiments, the netrin 1 compound is a peptide having an amino acid sequence that comprises, consists essentially of, or consists of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17. In some embodiments, the netrin 1 compound is about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long. In some embodiments, the netrin 1 compound is 8, 9, 10, or 11 amino acid residues long. In some embodiments, the netrin 1 compound is a peptide that comprises, consists essentially of, or consists of an amino acid sequence that has at least 90% sequence identity to SEQ ID NO: 9.

In some embodiments, daily administration of the one or more netrin-1 compounds results in a total body weight that is about 95-100% lower than a control. In some embodiments, daily administration of the one or more netrin-1 compounds: for at least about 4 weeks results in a total body weight that is about 20% lower than a control; for at least about 8 weeks results in a total body weight that is about 45% lower than a control; for at least about 12 weeks results in a total body weight that is about 65% lower than a control; or for at least about 16 weeks results in a total body weight that is about 90% lower than a control. In some embodiments, daily administration of the one or more netrin-1 compounds: for at least about 4 weeks results in a liver fat content that is about 10% lower than a control; for at least about 8 weeks results in a liver fat content that is about 25% lower than a control; for at least about 12 weeks results in a liver fat content that is about 35% lower than a control; or for at least about 16 weeks results in a liver fat content that is about 50% lower than a control. In some embodiments, daily administration of the one or more netrin-1 compounds results in about a 66-100% lower atherosclerotic lesions as compared to a control. In some embodiments, daily administration of the one or more netrin-1 compounds: for at least about 4 weeks results in atherosclerotic lesions that are about 10% lower than a control; for at least about 8 weeks results in atherosclerotic lesions that are about 25% lower than a control; for at least about 12 weeks results in atherosclerotic lesions that are about 35% lower than a control; or for at least about 16 weeks results in atherosclerotic lesions that are about 50% lower than a control. In some embodiments, daily administration of the one or more netrin-1 compounds results in about a 66-100% lower total cholesterol, triglycerides, and/or LDL levels as compared to a control. In some embodiments, daily administration of the one or more netrin-1 compounds: for at least about 4 weeks results in a total cholesterol level that is about 20% lower than a control; for at least about 8 weeks results in a total cholesterol level that is about 40% lower than a control; for at least about 12 weeks results in a total cholesterol level that is about 60% lower than a control; or for at least about 16 weeks results in a total cholesterol level that is about 80% lower than a control. In some embodiments, daily administration of the one or more netrin-1 compounds: for at least about 4 weeks results in an LDL level that is about 15% lower than a control; for at least about 8 weeks results in an LDL level that is about 30% lower than a control; for at least about 12 weeks results in an LDL level that is about 45% lower than a control; or for at least about 16 weeks results in an LDL level that is about 60% lower than a control. In some embodiments, the LDL level is the level of LDL cholesterol (LDL-C). In some embodiments, the one or more netrin-1 compounds are administered in a therapeutically effective amount. In some embodiments, the one or more netrin-1 compounds are administered in the form of a pharmaceutical composition. In some embodiments, the subject is mammalian, preferably human.

Both the foregoing general description and the following detailed description are exemplary and explanatory only and are intended to provide further explanation of the invention as claimed. Any accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1: Netrin-1 infusion prevented body weight increase and alleviated lesion formation in apoE−/− mice fed high fat diet. ApoE−/− mice were fed with high fat diet (42% fat) for 16 weeks, with or without netrin-1 infusion (15 ng/day). There were significant differences in body weight change and liver size (Panel A). Oil red-O staining was performed to observe lesion formation. Netrin-1 significantly abrogated lesion formation (Panels B & C).

FIG. 2: Netrin-1 infusion improved plasma lipid profile in apoE−/− mice fed high fat diet. Levels of plasma cholesterol and triglycerides were robustly decreased with netrin-1 infusion, while there was no difference in HDL-cholesterol levels.

FIG. 3: Netrin-1 and peptides are robustly lipid-lowering, anti-atherosclerotic and anti-obesity in high fat-fed apoE null mice. The apoE null male mice of 12-14 weeks were fed with high fat diet (42%, Harlan Laboratories) for 16 weeks, with or without infusion of netrin-1 (15 ng/day) or V1+V2 peptides. (Panels A-B) Aortic lesion formation robustly inhibited by netrin-1/V1+V2. ** p<0.01 vs. CTRL. (Panels C-F) Changes in plasma lipid profiles. ** p<0.01, *** p<0.001 vs. CTRL.

FIG. 4-FIG. 5: Netrin-1 infusion decreased accumulation of fatty streak at aortic root in apoE−/− mice fed high fat diet. Four out of four animals (=100%) of CTRL (FIG. 4), while only three out of seven animals (=43%) with netrin-1 infusion (FIG. 5) developed fatty streak. Black arrows indicate fatty streaks.

FIG. 6-FIG. 7: Netrin-1 infusion reduced macrophage infiltration at the aortic root. Immunohistochemistry using anti-Mac3 (CD107) antibody was performed to visualize macrophage accumulation. The most intense responsive areas of aortic roots were selected double-blindly. CTRL animals (FIG. 6) had stronger signals compare to netrin-1 infused animals (FIG. 7). White arrowheads indicate Mac3 positive cells.

FIG. 8: Netrin-1 infusion attenuated monocyte adhesion to endothelium via UNC5B. The monocytes-EC adhesion assay was performed using Calcein-AM labeling on monocytes (Panel A). The result shows netrin-1 treatment on ECs inhibited monocyte adhesion, but the effect was reduced by UNC5B antibody pre-treatment on monocytes (n=7, **p<0.01 vs CTRL). Shown in (Panel B) is Western blot analysis of monocyte in response to netrin-1 treatment. UNC5B cleavage level was increased in monocytes in the presence of netrin-1 (n=5, ***p<0.0001).

FIG. 9: Netrin-1 inhibited monocyte migration via UNC5B. Monocytes isolated from bone marrow were subjected to transwell migration assay in the presence or absence of UNC5B antibody. Netrin-1 inhibition of monocyte migration was reversed by antagonization of UNC5B.

FIG. 10: Netrin-1 upregulation by p47phox, and augmented inhibition of monocyte migration in p47phox dependent mice by netrin-1. Netrin-1 inhibition of monocyte migration was more prominent in p47phox deficient mice (upper panel). UNC5B expression was markedly upregulated in p47phox null mice (lower panel).

FIG. 11: Netrin-1 attenuates vascular smooth muscle cell (VSMC) migration in vitro and macrophage infiltration in vivo. VSMCs and ECs were co-cultured in and subjected to a transwell migration assay in the absence or presence of pharmacological inhibitors. Data indicate NO, cGMP, and p38 MAPK-dependent attenuation of VSMC migration by netrin-1. Images of tricuspid/aortic root with Mac-3 staining (data not shown) indicate macrophage infiltration was reduced by netrin-1 infusion in high fat fed apoE null mice.

FIG. 12: Netrin-1 infusion attenuated neointimal formation and restenosis in male and female subjects. Femoral artery was injured by the passing of a guide ware, and netrin-1 was infused via an osmotic minipump. (Panel A) and (Panel C) H&E images at Week 4 (Panel B) and (Panel D) grouped data of intima to media ratio. N=4-8, *p<0.01 for (Panel B), n=3-4 for (Panel D), Scale=100 μm.

### DETAILED DESCRIPTION OF THE INVENTION

Netrin-1 and netrin-1 peptides exhibit cardioprotective activity when administered to subjects. See PCT/US2011/038277; PCT/US2015/023248; Li & Cai (2015) Am J Physiol Cell Physiol 309:C100-106; and Nguyen & Cai (2006) PNAS USA 103: 6530-5, which are herein incorporated by reference in their entirety.

As disclosed herein, administration of netrin-1 compounds abolished weight increase and fatty liver, and reduced lesion formation. Administration of netrin-1 compounds also drastically reduced total cholesterol and triglyceride levels in subjects. Additionally, administration of netrin-1 compounds inhibited monocyte adhesion to endothelial cells (ECs), and increased UNC5B cleavage in monocytes. That is, netrin-1 compounds repel monocytes from endothelium and increase monocyte apoptosis, thereby indicating that netrin-1 compounds may prevent or inhibit plaque rupture and may therefore reverse the earliest stage of atherosclerosis lesion.

### Netrin-1 Compounds Reduce Body Fat, Fat Deposits, and Fatty Liver

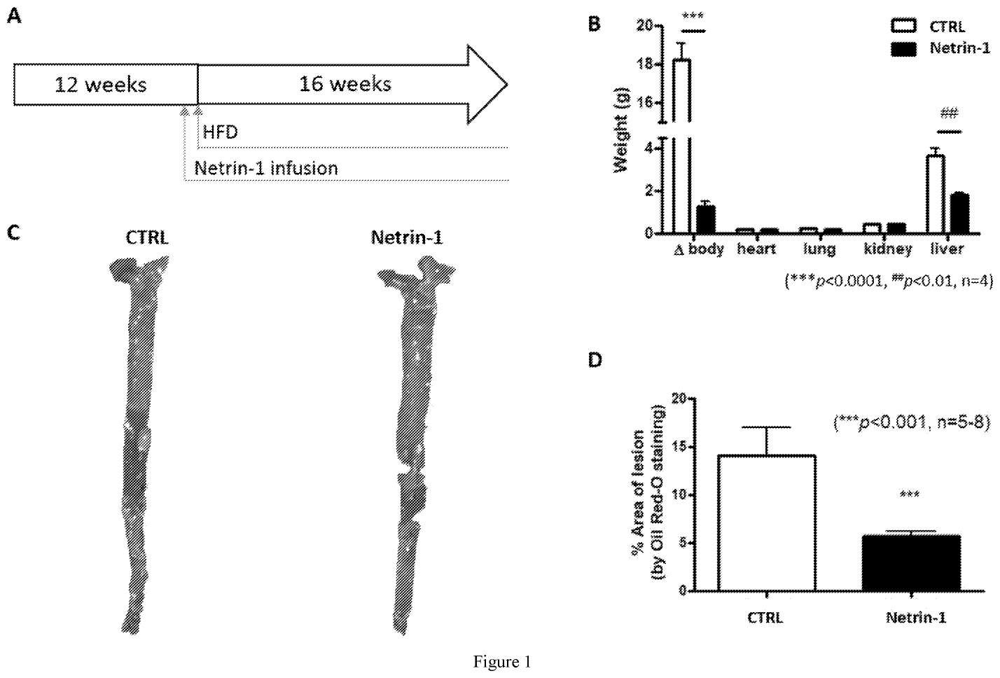

ApoE−/− mice develop hyperlipidemia and atherosclerosis even with normal chow diet as they age. ApoE−/− mice were divided into a netrin-1 treatment group and a control group (CTRL) and both were administered a high fat diet (HFD). As used herein, a “high fat diet” refers to a diet in which at least about 40% of a subject's daily caloric intake are fat calories (i.e., calories from fat). The netrin-1 group was infused with netrin-1 one day in advance of commencement of the HFD and netrin-1 infusion continued for 16 weeks (FIG. 1, Panel A), whereas the control group was fed the HFD without any netrin-1 infusions. After 16 weeks of HFD, the appearance of two groups of mice are visibly different.

As shown in FIG. 1, Panel B, the body weight increase in the control group was significant, while netrin-1 group stayed unchanged. The control group exhibited higher fat amounts and fatty liver similar to subjects who have a higher risk of atherosclerosis. Therefore, netrin-1 compounds inhibit and/or reduce body fat and fat deposits. netrin-1 compounds also inhibits, reduces, and/or treats fatty liver.

### Netrin-1 Compounds Reduce Atherosclerotic Lesions

To visualize any atherosclerotic lesions in the aortas of the subjects, oil red-O staining was performed. Compared to the control group, the netrin-1 group exhibited significantly lower occurrences of lesions (FIG. 1, Panels C, D). Thus, netrin-1 compounds inhibit, reduce, and/or treat atherosclerotic lesions.

### Netrin-1 Compounds Reduce Hyperlipidemia

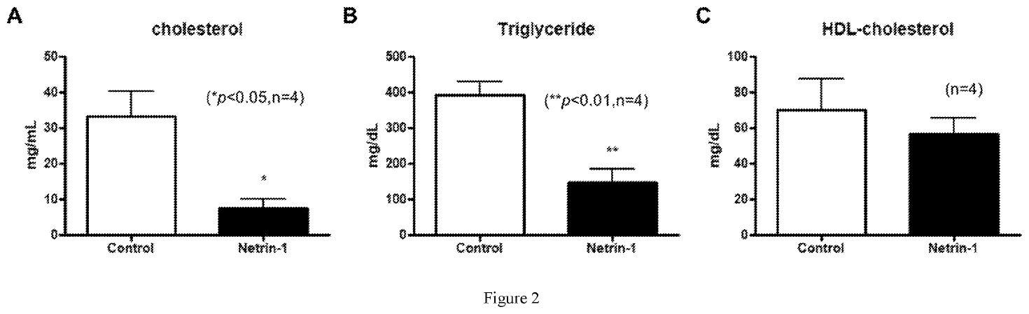

The typical lipid profile of subjects suffering from atherosclerosis is (a) a high level of total cholesterol, low-density lipoprotein cholesterol (LDL), and triglycerides, and (b) a low level of high-density lipoprotein cholesterol (HDL). Thus, the plasma lipid profiles of the subjects were examined. As shown in FIG. 2, Panels A-C, compared to the control group, the netrin-1 group had significantly lower levels of total cholesterol and triglycerides, while HDL-cholesterol levels were similar. Therefore, netrin-1 compounds significantly reduce total cholesterol and LDLs in subjects.

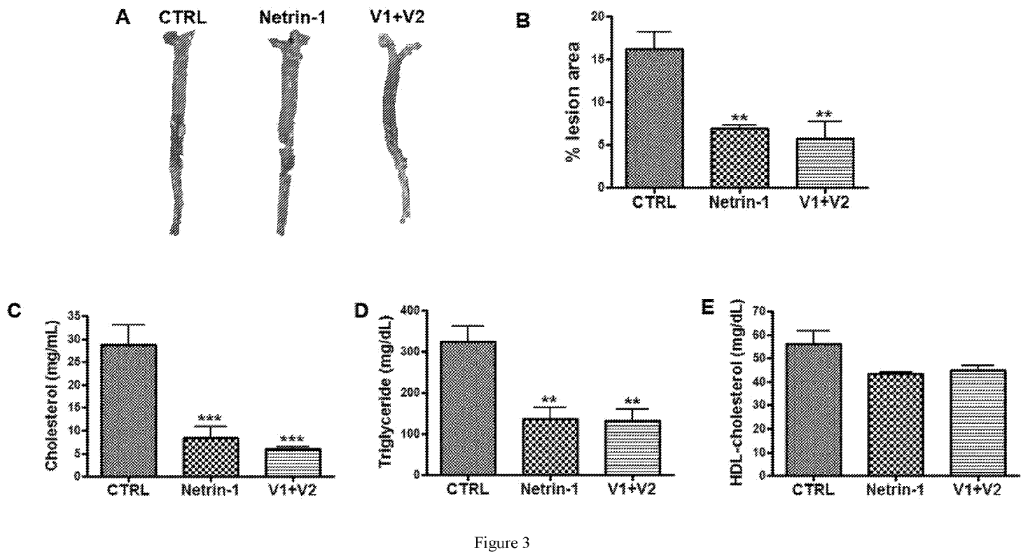

Administration of netrin-1 peptides, V1 and V2, also resulted in lower aortic lesions (FIG. 3, Panels A, B). While V1 and V2 resulted in triglyceride (FIG. 3, Panel D) and HDL-cholesterol (FIG. 3, Panel E) levels that were similar to that provided by netrin-1, V1 and V2 resulted in slightly lower total cholesterol levels (FIG. 3, Panel C) compared to netrin-1.

### Netrin-1 Compounds Reduce Fat Deposits in Arteries

Hypercholesterolemia induced by apoE knockout and accelerated by HFD, results in a formation of fatty streak, which is the first grossly visible lesion to be formed during the development of atherosclerosis. Fatty streak accumulation triggers the subsequent vicious cycle of pathogenesis such as fibrosis and plaque rupture. The fatty streak is most frequently seen at the root of aorta which can be identified by the unique structure of tricuspid valve.

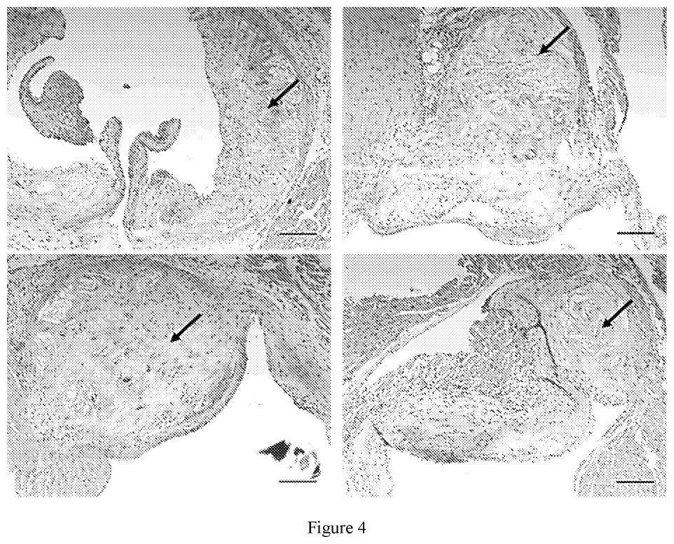

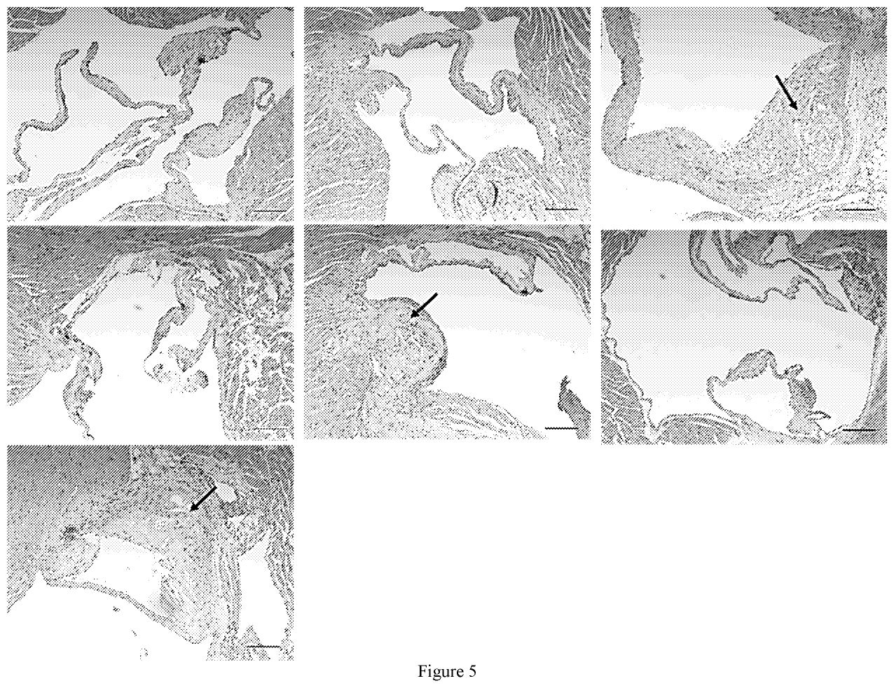

The aortic roots of both control and netrin-1 groups were screened for fatty streak (FIG. 4, FIG. 5). All subjects of the control group exhibited a large fatty streak. On the other hand, only three out of seven (43%) subjects of the netrin-1 group developed a fatty streak (vs. 100% in HFD fed apoE mice without netrin-1 treatment), which fatty streaks were comparatively smaller than that of the control group. Therefore, netrin-1 compounds inhibit and/or reduce fatty streak incidence and accumulation in the aortic root. As such, netrin-1 compounds can be used to inhibit or reduce pathologies resulting from fatty streak accumulation, e.g., fibrosis and/or plaque rupture.

### Netrin-1 Compounds Inhibit Arterial Macrophage Infiltration

Under atherosclerotic environment, circulating monocytes are retained to endothelium and differentiate into macrophages in the subendothelial region. The differentiated macrophages react with modified LDLs such as ox-LDLs to form foam cells. The foam cells capture various immune cells such as T-cells, dendritic cells, and mast cells. This reaction further recruits more inflammatory cells and modified LDLs, leading the initiation and fatty streak phase of atherosclerotic lesions. Therefore, macrophage accumulation is an early sign of atherogenesis.

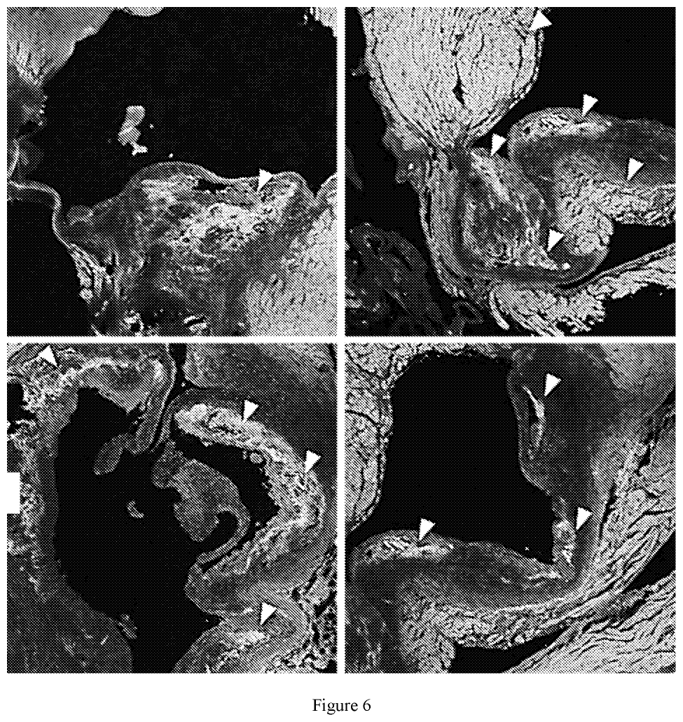

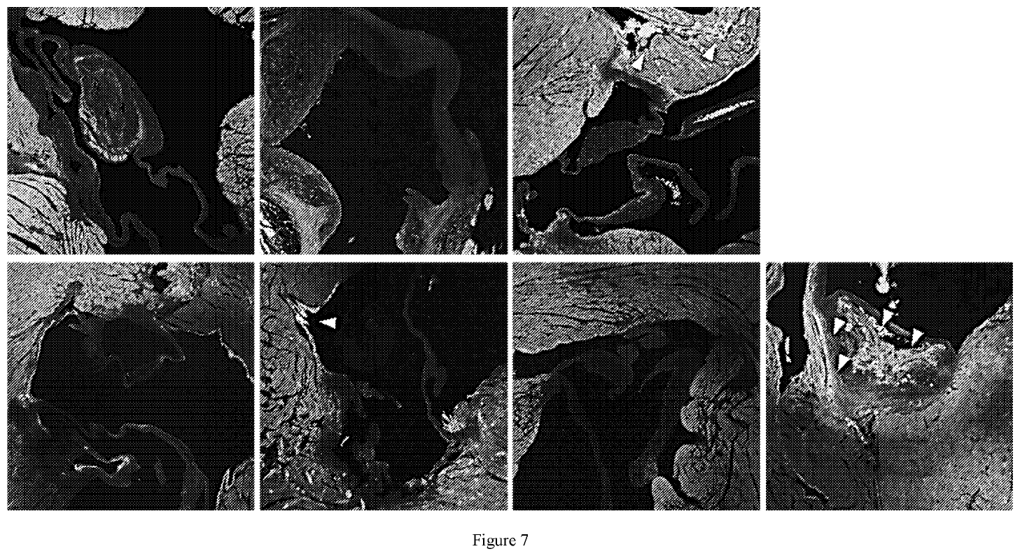

Immunochemical staining was used to evaluate macrophage accumulation in subjects of the control and netrin-1 groups. As shown in FIG. 6 and FIG. 7, accumulation of Mac-3 (CD107) positive cells was significantly less in netrin-1 infused subjects as compared to controls. These results are consistent to the fatty streak formation data showed in FIG. 5. Therefore, netrin-1 inhibits or reduces macrophage accumulation, formation of foam cells, fatty streak formation and accumulation, and atherogenesis.

### Netrin-1 Compounds Inhibit Monocyte Adhesion to Endothelial Cells Via UNC5B

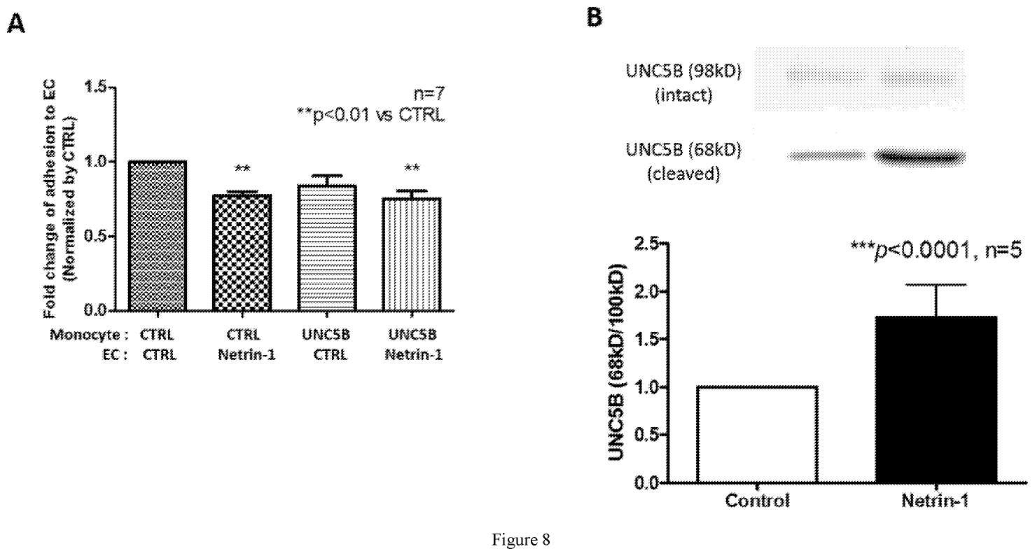

Monocytes, the major source of inflammatory response in atherosclerosis, express UNC5B as a dominant receptor of netrin-1. UNC5B receptor is a repulsive receptor of netrin-1. As monocytes expressing UNC5B may be repulsed by netrin-1 expressed in endothelial cells (ECs), a monocyte-EC adhesion assay was performed. FIG. 8, Panel A, shows that treating ECs with netrin-1 results in a 23% inhibition of monocyte adhesion. Netrin-1 treatment of ECs had no effect on monocyte adhesion on monocytes that were pretreated with UNC5B antibody to mask the receptors. These results suggest that the anti-adhesion effect of netrin-1 is mediated by UNC5B.

When netrin-1 binds to UNC5B, the intracellular death domain of UNC5B is cleaved and the cell expressing UNC5B proceeds to apoptosis. Therefore, the cleavage of UNC5B in response to netrin-1 treatment was assayed. The results show that netrin-1 significantly increases cleavage of UNC5B (FIG. 8, Panel B), thereby indicating that netrin-1 binding increases death domain activation in monocytes.

These results suggest that the anti-inflammatory effect of netrin-1 is, at least, partially mediated by anti-attraction and pro-apoptosis of monocytes via UNC5B expression.

Therefore, the present invention provides netrin-1 compounds and compositions for treating, reducing, or inhibiting a hyperlipidemia condition in subjects. As used herein, a “hyperlipidemia condition” refers to conditions resulting from or related to hyperlipidemia. Such hyperlipidemia conditions include hyperlipidemia, hypercholesterolemia, obesity, fatty liver, fatty deposits in arteries, arterial macrophage infiltration, atherosclerotic lesions, monocyte migration, vascular smooth muscle cell migration, monocyte adhesion to endothelial cells, neointimal formation, and restenosis. In some embodiments, the subject has been diagnosed with a hyperlipidemia condition. In some embodiments, the subject is in need of treatment for a hyperlipidemia condition. In some embodiments, the subject has been diagnosed as having a hyperlipidemia condition. In some embodiments, the hyperlipidemia condition is hyperlipidemia, hypercholesterolemia, obesity, fatty liver, fatty deposits in arteries, arterial macrophage infiltration, atherosclerotic lesions, monocyte migration, vascular smooth muscle cell migration, or monocyte adhesion to endothelial cells.

As used herein, “netrin-1 compounds” refer to the full-length human netrin-1 protein (GI 148613884), proteins having at least 90%, at least 91%, at least 92%, at least 93%, at least 94%, at least 95%, at least 96%, at least 97%, at least 99% sequence identity to the full-length human netrin-1 protein, and netrin-1 peptides. Netrin-1 compounds may or may not exhibit the same or similar activity as the full-length human netrin-1 protein. Nevertheless, in some embodiments, a netrin-1 compound, e.g., a netrin-1 peptide, exhibits substantially similar activity as the full-length human netrin-1 protein. In some embodiments, a netrin-1 compound, e.g., a netrin-1 peptide, exhibits significantly better activity and/or a different biological activity as the full-length human netrin-1 protein.

As used herein, a “netrin-1 peptide” refers to a peptide or protein that comprises, consists essentially of, or consists of SEQ ID NO: 1 as follows:

| | |
|---|---|
| X1-X2-X3-C-X4-X5-X6-X7-T-X8-G | (SEQ ID NO: 1) |

wherein

X1 is Ala, Asn, Cys, D-Cys, Ser, or Thr, preferably X1 is Cys, Ser, or Thr;

X2 is present or absent, and if present, X2 is Ala, Asp, Ile, Leu, Met, Phe, Pro, Trp, or Val, preferably X2 is Leu or Pro;

X3 is present or absent, and if present, X3 is Asn, Arg, Asp, Cys, Gln, Glu, Gly, Ser, Thr, or Tyr, preferably X3 is Asn or Asp;

X4 is Arg, His, or Lys, preferably X4 is Arg or Lys;

X5 is Arg, Asp, Glu, His, Lys, Phe, Trp, or Tyr, preferably X5 is Asn, Asp, or His;

X6 is Asn, Cys, Gln, Gly, Ser, Thr, Tyr, or Val, preferably X6 is Asn or Gly;

X7 is present or absent, and if present, X7 is Asn, Gly, His, Ile, Thr, or Val, preferably X7 is Val; and

X8 is present or absent, and if present, X8 is Ala, Asn, Ile, Leu, Met, Phe, Pro, Thr, Trp, or Val, preferably X8 is Ala; and

wherein X2, X3, or both X2 and X3 are present.

In some embodiments, when X1 is Cys, it is covalently attached to either the cysteine residue at the fourth amino acid position via a disulfide bond or an ethylene oxide compound. In some embodiments, when X1 is D-Cys, the glycine residue at the 10^{th} amino acid position is a D-amino acid, the last amino acid residue at the C-terminal end is a D-amino acid, or both the glycine residue at the 10^{th} amino acid position and the last amino acid residue at the C-terminal end are D-amino acids.

In some embodiments, the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG.

In some embodiments, the netrin-1 peptides are about 8-60, about 8-55, about 8-50, about 8-45, about 8-40, about 8-35, about 8-30, about 8-20, about 8-15, about 8-12, 8-11, about 9-60, about 9-55, about 9-50, about 9-45, about 9-40, about 9-35, about 9-30, about 9-20, about 9-15, about 9-12, or 9-11 amino acid residues long. In some embodiments, the netrin-1 peptides are 8, 9, 10, or 11 amino acid residues long.

As used herein, a peptide that “comprises” a given sequence means that the peptide may include additional amino acid residues, amino acid isomers, and/or amino acid analogs at the N-terminus, the C-terminus, or both. The additional residues may or may not change the activity or function of the given sequence, i.e., the peptide having the additional residues, isomers, or analogs may have a different activity or function as compared to the given sequence itself (without the additional residues, isomers, or analogs). As used herein, a peptide that “consists essentially of” a given sequence means that the peptide may include additional amino acid residues, amino acid isomers, and/or amino acid analogs at the N-terminus, the C-terminus, or both, so long as they do not materially change the function or activity of the given sequence, i.e., the peptide having the additional residues, isomers, or analogs has an activity and function that are substantially similar to that of the given sequence itself. As used herein, a peptide that “consists of” a given sequence means that the peptide does not include additional amino acid residues, amino acid isomers, and/or amino acid analogs at either the N-terminus or the C-terminus.

In some embodiments, netrin-1 compounds may be isolated. As used herein, an “isolated” compound refers to a compound which is isolated from its native environment. For example, an isolated peptide is one which does not have its native amino acids, which correspond to the full-length polypeptide, flanking the N-terminus, C-terminus, or both. For example, an isolated V1-9aa peptide refers to a peptide having amino acid residues (304-312 aa) of V1, which may have non-native amino acids at its N-terminus, C-terminus, or both, but does not have a proline amino acid residue following its 9^{th} amino acid residue at the C-terminus, or a valine amino acid residue immediately preceding the cysteine amino acid residue at its N-terminus, or both. As another example, an isolated peptide can be one which is immobilized to a substrate with which the peptide is not naturally associated. As a further example, an isolated peptide can be one which is linked to another molecule, e.g., a PEG compound, e.g., mPEG, with which the peptide is not naturally associated.

In some embodiments, netrin-1 compounds may comprise one or more natural amino acids, unnatural amino acids, or a combination thereof. The amino acid residues of the peptide may be D-isomers, L-isomers, or both. The peptide may be composed of α-amino acids, β-amino acids, natural amino acids, non-natural amino acids, amino acid analogs, or a combination thereof. Amino acid analogs include β-amino acids and amino acids where the amino or carboxy group is substituted by a similarly reactive group (e.g., substitution of the primary amine with a secondary or tertiary amine, or substitution of the carboxy group with an ester).

Examples of β-amino acid analogs include cyclic β-amino acid analogs; β-alanine; I-β-phenylalanine; I-1,2,3,4-tetrahydro-isoquinoline-3-acetic acid; I-3-amino-4-(1-naphthyl)-butyric acid; I-3-amino-4-(2,4-dichlorophenyl)butyric acid; I-3-amino-4-(2-chlorophenyl)-butyric acid; I-3-amino-4-(2-cyanophenyl)-butyric acid; I-3-amino-4-(2-fluorophenyl)-butyric acid; I-3-amino-4-(2-furyl)-butyric acid; I-3-amino-4-(2-methylphenyl)-butyric acid; I-3-amino-4-(2-naphthyl)-butyric acid; I-3-amino-4-(2-thienyl)-butyric acid; I-3-amino-4-(2-trifluoromethylphenyl)-butyric acid; I-3-amino-4-(3,4-dichlorophenyl)butyric acid; I-3-amino-4-(3,4-difluorophenyl)butyric acid; I-3-amino-4-(3-benzothienyl)-butyric acid; I-3-amino-4-(3-chlorophenyl)-butyric acid; I-3-amino-4-(3-cyanophenyl)-butyric acid; I-3-amino-4-(3-fluorophenyl)-butyric acid; I-3-amino-4-(3-methylphenyl)-butyric acid; I-3-amino-4-(3-pyridyl)-butyric acid; I-3-amino-4-(3-thienyl)-butyric acid; I-3-amino-4-(3-trifluoromethylphenyl)-butyric acid; I-3-amino-4-(4-bromophenyl)-butyric acid; I-3-amino-4-(4-chlorophenyl)-butyric acid; I-3-amino-4-(4-cyanophenyl)-butyric acid; I-3-amino-4-(4-fluorophenyl)-butyric acid; I-3-amino-4-(4-iodophenyl)-butyric acid; I-3-amino-4-(4-methylphenyl)-butyric acid; I-3-amino-4-(4-nitrophenyl)-butyric acid; I-3-amino-4-(4-pyridyl)-butyric acid; I-3-amino-4-(4-trifluoromethylphenyl)-butyric acid; I-3-amino-4-pentafluoro-phenylbutyric acid; I-3-amino-5-hexenoic acid; I-3-amino-5-hexynoic acid; I-3-amino-5-phenylpentanoic acid; I-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; I-3-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Examples of amino acid analogs of alanine, valine, glycine, and leucine include α-methoxyglycine; α-allyl-L-alanine; α-aminoisobutyric acid; α-methyl-leucine; β-(1-naphthyl)-D-alanine; β-(1-naphthyl)-L-alanine; O-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Examples of amino acid analogs of arginine and lysine include citrulline; L-2-amino-3-guanidinopropionic acid; L-2-amino-3-ureidopropionic acid; L-citrulline; Lys(Me)2-OH; Lys(N3)-OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)2-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Examples of amino acid analogs of aspartic and glutamic acids include α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(Oall)-OH; L-Asu(OtBu)-OH; and pyroglutamic acid.

Examples of amino acid analogs of cysteine and methionine include Cys(farnesyl)-OH, Cys(farnesyl)-Ome, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cy steine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Examples of amino acid analogs of proline include 3,4-dehydro-proline, 4-fluoro-proline, cis-4-hydroxy-proline, thiazolidine-2-carboxylic acid, and trans-4-fluoro-proline.

Examples of amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Examples of amino acid analogs of tryptophan include α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, netrin-1 compounds may comprise one or more non-essential amino acids. A non-essential amino acid residue can be a residue that can be altered from the wild-type sequence of a polypeptide without abolishing or substantially altering its essential biological or biochemical activity (e.g., receptor binding or activation).

In some embodiments, netrin-1 compounds may comprise one or more conservative amino acid substitutions. In some embodiments, a conservative amino acid substitution is one in which the amino acid residue is replaced with an amino acid residue having a side chain. Amino acids with basic side chains include Arg, His, and Lys amino acids with acidic side chains include Asp and Glu, amino acids with uncharged polar side chains include Asn, Cys, Gln, Gly, Ser, Thr, and Tyr, amino acids with nonpolar side chains include Ala, Ile, Leu, Met, Phe, Pro, Trp, and Val, amino acids with I-branched side chains include Ile, Thr, and Val, and amino acids with aromatic side chains include His, Phe, Trp, and Tyr. In some embodiments, a conservative amino acid substitution is a very highly conserved substitution, a highly conserved substitution, or a conserved substitution as set forth in the following table:

| | Very Highly- | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

As disclosed herein, netrin-1 compounds are shown to effectively treat hyperlipidemia conditions. Therefore, in some embodiments, one or more netrin-1 compounds may be used to treat, inhibit, or reduce a hyperlipidemia condition in a subject. In some embodiments, the subject to be treated with one or more netrin-1 compounds suffers from a hyperlipidemia condition. In some embodiments, the subject has been diagnosed as having a hyperlipidemia condition. In some embodiments, the subject exhibits symptoms associated with one or more hyperlipidemia conditions. In some embodiments, the subject is in need of treatment for a hyperlipidemia condition. Subjects who are “in need of treatment for a hyperlipidemia condition” include those who are at risk of a hyperlipidemia condition, suffer from a hyperlipidemia condition, exhibit symptoms of a hyperlipidemia condition, have high cholesterol levels, or have high LDL levels.

### Netrin-1 Compounds and Compositions and Administration

Administration of one or more netrin-1 compounds can be accomplished by direct administration or accomplished by administering one or more nucleic acid molecules which encode the one or more netrin-1 compounds.

In some embodiments, a therapeutically effective amount of one or more netrin-1 compounds are administered to a subject. As used herein, a “therapeutically effective amount” refers to an amount that may be used to treat, alleviate, ameliorate, prevent, or inhibit a given disease or condition, such as a hyperlipidemia condition or a symptom thereof, in a subject as compared to a control, such as a placebo. For example, in some embodiments, a therapeutically effective amount is an amount which has a beneficial effect in a subject, e.g., reduces high levels of cholesterol and/or high levels of LDLs, in the subject as compared to a normal control and/or a negative control. In some embodiments, a therapeutically effective amount is an amount which inhibits or reduces signs and/or symptoms of a hyperlipidemia condition, such as high levels of cholesterol and/or high levels of LDLs, as compared to a normal control and/or a negative control. The skilled artisan will appreciate that certain factors may influence the amount required to effectively treat a subject, including the degree of the given disease or condition, previous treatments, the general health and age of the subject, and the like. Nevertheless, therapeutically effective amounts may be readily determined by methods in the art. In some embodiments, a therapeutically effective amount of a netrin-1 compound according to the present invention ranges from about 1 ng/kg to about 100 mg/kg body weight, about 0.001 mg/kg to about 100 mg/kg body weight, about 0.01 mg/kg to about 10 mg/kg body weight, about 0.01 mg/kg to about 5 mg/kg body weight, about 0.01 mg/kg to about 3 mg/kg body weight, about 0.01 mg/kg to about 2 mg/kg, about 0.01 mg/kg to about 1 mg/kg, or about 0.01 mg/kg to about 0.5 mg/kg body weight. In some embodiments, about 1 ng/kg to about 25 ng/kg, preferably about 10 ng/kg to about 20 ng/kg, and more preferably about 15 ng/kg, body weight of one or more netrin-1 compounds are administered daily to a subject over a given period, e.g., about 3 weeks. In some embodiments, the administration is subcutaneous. In some embodiments, the mode of administration provides a controlled release of the one or more netrin-1 compounds. In some embodiments, the one or more netrin-1 compounds may be administered using a subcutaneously implanted drug delivery device such as an osmotic mini-pump. In some embodiments, the one or more netrin-1 compounds are administered subcutaneously, e.g., by injection, in the form of a sustained release composition. See, e.g., Schaefer et al. (2016) Journal of Drug Delivery 2016: 2407459.

It should be noted that treatment of a subject with a therapeutically effective amount may be administered as a single dose or as a series of several doses. The dosages used for treatment may increase or decrease over the course of a given treatment. Optimal dosages for a given set of conditions and a given subject may be ascertained by those skilled in the art using dosage-determination tests and/or diagnostic assays in the art. Dosage-determination tests and/or diagnostic assays may be used to monitor and adjust dosages during the course of treatment. In some embodiments, the one or more netrin-1 compounds are administered in the form of a composition.

In some embodiments, the compositions comprise, consist essentially of, or consist of one or more netrin-1 compounds. As used herein, a composition “comprising” one or more netrin-1 compounds means that the composition may contain other compounds, including proteins that are not netrin-1 compounds (e.g., netrin-1 compounds). As used herein, a composition “consisting essentially of” one or more netrin-1 compounds means that the composition may comprise proteins in addition to the netrin-1 compounds so long as the additional proteins do not materially change the activity or function of the netrin-1 compounds that are contained in the composition. As used herein, a composition “consisting of” one or more netrin-1 compounds means that the composition does not contain proteins in addition to the one or more netrin-1 compounds. Compositions consisting of one or more netrin-1 compounds may comprise ingredients other than proteins, e.g., pharmaceutically acceptable carriers, surfactants, preservatives, etc. In some embodiments, compositions consisting of one or more netrin-1 compounds may contain insignificant amounts of contaminants, which may include peptide contaminants, e.g., smaller fragments of the one or more netrin-1 compounds, which may result from, for example, the synthesis of the one or more netrin-1 compounds, subsequent processing, storage conditions, and/or protein degradation.

In some embodiments, the compositions may comprise, consist essentially of, or consist of one or more purified netrin-1 compounds. As used herein, a “purified” netrin-1 compound means that an amount of the macromolecular components that are naturally associated with the netrin-1 compound have been removed from the netrin-1 compound. As used herein, a composition comprising, consisting essentially of, or consisting of one or more purified netrin-1 compounds means that the composition does not contain an amount of the macromolecular components that are naturally associated with the one or more netrin-1 compounds and/or the reagents used to synthesize the netrin-1 compounds. In some embodiments, the amount removed from the one or more netrin-1 compounds (or is not present in the composition) is at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components and/or reagents. In some embodiments, the composition is free of at least about 60%, at least about 70%, at least about 75%, at least about 80%, at least about 85%, at least about 90%, at least about 95%, at least about 96%, at least about 96%, at least about 97%, at least about 98%, at least about 99%, or 100% of the macromolecular components naturally associated with the one or more netrin-1 compounds and/or the reagents used to synthesize the one or more netrin-1 compounds. In some embodiments, the compositions of the present invention consist solely of one or more netrin-1 compounds, e.g., the one or more netrin-1 compounds in a solid or crystalized form.

In some embodiments, compositions according to the present invention include one or more netrin-1 compounds and a pharmaceutically acceptable carrier. The term “pharmaceutically acceptable carrier” as used herein refers to a carrier or diluent, which are added to a composition by the hand of man, that is generally non-toxic to an intended recipient and does not significantly inhibit activity of the one or more netrin-1 compounds included in the composition. In some embodiments, compositions according to the present invention may include one or more excipients, diluents, auxiliaries, preservatives, solubilizing agents, buffers, thickening agents, gelling agents, foaming agents, surfactants, binders, suspending agents, disintegrating agents, wetting agents, solvents, plasticizers, fillers, colorants, dispersants, flavoring agents, and/or the like known in the art.

A composition according to the present invention generally includes about 0.1-99% of one or more netrin-1 compounds. In some embodiments, a composition according to the present invention includes one or more netrin-1 peptides and a full-length netrin-1 protein, such as the full-length human netrin-1 protein. In some embodiments, the compositions are synergistic compositions, e.g., compositions comprising a first netrin-1 compound and a second netrin-1 compound in synergistic amounts.

In some embodiments, one or more netrin-1 compounds are included in a composition of the present invention in the form of a free acid or free base. In some embodiments, one or more netrin-1 compounds are included in a composition in the form of a pharmaceutically acceptable salt such as an acid or base addition salt. A pharmaceutically acceptable salt refers to any salt form of the one or more netrin-1 compounds that is generally non-toxic to an intended recipient and does not significantly inhibit activity of the one or more netrin-1 compounds or other active agent included in the composition. In some embodiments, the one or more netrin-1 compounds are provided in the form of a hydrate or a prodrug.

A composition including one or more netrin-1 compounds may be administered by a systemic route and/or by a local route. Suitable routes of administration illustratively include intravenous, oral, buccal, parenteral, intrathecal, intracerebroventricular, intraperitoneal, intracardiac, intraarterial, intravesical, ocular, intraocular, rectal, vaginal, subcutaneous, intradermal, transdermal, intramuscular, topical, intranasal, and transmucosal. In some embodiments, the one or more netrin-1 compounds and compositions thereof are administered intravenously or by intraventricular injection.

In some embodiments, the netrin-1 compounds and compositions according to the present invention may be modified using methods and compositions known in the art to improve their biological half-life, stability, efficacy, bioavailability, bioactivity, or a combination thereof. For example, in some embodiments, the netrin-1 compounds may be subjected to cyclization to result in a cyclic peptide which is resistant to proteolytic degradation. Cyclization may be carried out between side chains or ends of the peptide sequences through disulfide bonds, lanthionine, dicarba, hydrazine, or lactam bridges using methods known in the art.

In some embodiments, the netrin-1 compounds may be conjugated to a molecule such as vitamin B12, a lipid, or an ethylene oxide compound, e.g., polyethylene glycol (PEG), polyethylene oxide (PEO), and polyoxyethylene (POE), methoxypolyethylene glycol (MPEG), monomethoxypolyethylene glycol (mPEG), diethylene glycol (mini-PEG), and the like. The ethylene oxide compound may be further functionalized with, for example, amine binding terminal functional groups such as N-hydroxysuccinimide esters, N-hydroxysuccinimide carbonates, and aliphatic aldehyde, or thiol binding groups such as maleimide, pyridyl disulphides, and vinyl sulfonates. Since amino groups (α-amino and ε-lysine amino) and cysteine residues are well suited for conjugation, the netrin-1 compounds may further include one or more amino acid residues for conjugation to an ethylene oxide molecule or a carrier compound known in the art. The pharmacokinetic and pharmacodynamic properties of a conjugated peptide may be further modified by the use of a particular linker. For example, propyl and amyl linkers can be used to provide a conjugate having a loose conformation whereas a phenyl linker may be used to provide a denser conformation as well as shield domains adjacent to the C-terminus. It is noted that dense conformations are generally more efficient in maintaining bioactivity, prolonging plasma half-life, lowering proteolytic sensitivity, and immunogenicity relative to loose conformations.

In some embodiments, the netrin-1 compounds may be hyperglycosylated using methods known in the art, e.g., in situ chemical reactions or site-directed mutagenesis. Hyperglycosylation may result in either N-linked or O-linked protein glycosylation. The clearance rate of a given netrin-1 compound may be optimized by the selection of the particular saccharide. For example, polysialic acid (PSA) is available in different sizes and its clearance depends on type and molecular size of the polymer. Thus, for example, PSAs having high molecular weights may be suitable for the delivery of low-molecular-weight netrin-1 compounds, and PSAs having low molecular weights may be suitable for the delivery of netrin-1 compounds having high molecular weights. The type of saccharide can be used to target the netrin-1 compound to a particular tissue or cell. For example, netrin-1 compounds conjugated with mannose can be recognized by mannose-specific lectins, e.g., mannose receptors and mannan-binding proteins, and are taken up by the liver. In some embodiments, the netrin-1 compounds may be hyperglycosylated to improve their physical and chemical stability under different environmental conditions, e.g., to inhibit inactivation under stress conditions and reduce aggregation resulting from production and storage conditions.

In some embodiments, a drug delivery system, such as microparticles, nanoparticles (particles having sizes ranging from 10 to 1000 nm), nanoemulsions, liposomes, and the like, may be used to provide protection of sensitive proteins, prolong release, reduce administration frequency, increase patient compliance, and control plasma levels. Various natural or synthetic microparticles and nanoparticles, which may be biodegradable and/or biocompatible polymers, may be used. Microparticles and nanoparticles can be fabricated from lipids, polymers, and/or metal. Polymeric microparticles and nanoparticles may be fabricated from natural or synthetic polymers, such as starch, alginate, collagen, chitosan, polycaprolactones (PCL), polylactic acid (PLA), poly (lactide-co-glycolide) (PLGA), and the like. In some embodiments, the nanoparticles are solid lipid nanoparticles (SLNs), carbon nanotubes, nanospheres, nanocapules, and the like. In some embodiments, the polymers are hydrophilic. In some embodiments, the polymers are thiolated polymers.

Since the rate and extent of drug release from microparticles and nanoparticles may depend on the composition of polymer and fabrication methods one may select a given composition and fabrication method, e.g., spray drying, lyophilization, microextrusion, and double emulsion, to confer a desired drug release profile. Since peptide fragments incorporated in or on microparticles or nanoparticles may be prone to denaturation at aqueous-organic interface during formulation development, different stabilizing excipients and compositions can be used to prevent aggregation and denaturation. For example, PEG and sugars, e.g., PEG (MW 5000) and maltose with α-chymotrypsin, may be added to the composition to reduce aggregation and denaturation. Additionally, chemically modified peptide fragments, e.g., conjugated peptide fragments and hyperglycosylated peptide fragments, may be employed.

Protein stability can also be achieved by the selected fabrication method. For example, to prevent degradation at aqueous-organic interface, non-aqueous methodology called ProLease® technology may be used. Peptide fragments in solid state can also be encapsulated using solid-in-oil-in-water (s/o/w) methods, e.g., spray- or spray-freeze-dried peptide fragments or peptide-loaded solid nanoparticles can be encapsulated in microspheres using s/o/w methods. Hydrophobic ion-pairing (HIP) complexation may be used to enhance protein stability and increase encapsulation efficiency into microparticles and nanoparticles. In hydrophobic ion-pairing (HIP) complexation, ionizable functional groups of a peptide are complexed with ion-pairing agents (e.g., surfactant or polymer) containing oppositely charged functional groups leading to formation of HIP complex where hydrophilic protein molecules exist in a hydrophobic complex form.

In some embodiments, liposomes of either synthetic or natural origin and various sizes, e.g., 20 nm to several hundred micrometers, may be used to deliver peptide fragments. Depending on the preparation method, the liposomes can be small unilamellar vesicles (25-50 nm), large unilamellar vesicles (100-200 nm), giant unilamellar vesicles (1-2 μm), and multilamellar vesicles (MLV; 1 μm-2 μm). The peptide fragments being delivered can be either encapsulated into liposomes or adsorbed on the surface. The size and surface properties of liposomes may be optimized for a desired result. For example, unilamellar and multilamellar liposomes provide sustained release from several hours to days after intravascular administration. The prolonged drug release can be achieved by multivesicular liposomes, also known as DepoFoam® technology. Unlike ULV and MLV, multivesicular liposomes are composed of nonconcentric multiple aqueous chambers surrounded by a network of lipid layers which confers an increased level of stability and longer duration of drug release. The liposomes may be further modified to achieve a desired result. For example, the liposomes may be PEGylated or have other surface modifications in order to interfere with recognition and uptake by the reticuloendothelial system and provide increased circulation times.

Exemplary liposomes suitable for use according to the present invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV).

The liposomes may comprise additional lipids, e.g., carrier lipids, including palmitoylphosphatidylcholine (DPPC), phosphatidylcholine (PC; lecithin), phosphatidic acid (PA), phosphatidylglycerol (PG), phosphatidylethanolamine (PE), phosphatidylserine (PS), di stearoylphosphatidylcholine (DSPC), dimyristoylphosphatidylcholine (DMPC), dipalmitoylphosphatidyglycerol (DPPG), di stearoylphosphatidyglycerol (DSPG), dimyristoylphosphatidylglycerol (DMPG), dipalmitoylphosphatidic acid (DPPA); dimyristoylphosphatidic acid (DMPA), di stearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE), and the like, or combinations thereof. In some embodiments, the liposomes further comprise a sterol (e.g., cholesterol).

In some embodiments, micelles may be used to deliver the netrin-1 compounds. Phospholipids such as DSPE-PEG, co-polymeric systems PEG-PE, PLA-PEG and hyperbranched poly([amine-ester]-co-[d,l-lactide]) and polyion complexes may be used to increase stability and pharmacokinetics.

Thermosensitive gels may be used to deliver the netrin-1 compounds. Thermoreversible block copolymers comprising PEG, PCL, PLA, poly(glycolide), PLGA, poly (N-isopropylacrylamide), polyethylene oxide, chitosan, and the like may be used to provide controlled release of the peptide fragments. Examples of thermosensitive gels include PLGA-PEG-PLGA triblock copolymer gels and Pluronic F-127 (PF127). Polyelectrolyte complexes and/or PEGylation may be used to provide sustained release of proteins from the gels. Microparticles and/or nanoparticles may also be used in combination with gels to provide sustained drug delivery.

Netrin-1 compounds may be chemically synthesized, or recombinantly expressed in a cell system or a cell-free system. Synthetic methods include liquid-phase synthesis, solid-phase synthesis, and microwave assisted peptide synthesis. The peptide fragments may be modified by acylation, alkylation, amidation, arginylation, polyglutamylation, polyglycylation, butyrylation, gamma-carboxylation, glycosylation, malonylation, hydroxylation, iodination, nucleotide addition (e.g., ADP-rib osylation), oxidation, phosphorylation, adenylylation, propionylation, S-glutathionylation, S-nitrosylation, succinylation, sulfation, glycation, palmitoylation, myristoylation, isoprenylation or prenylation (e.g., farnesylation or geranylgeranylation), glypiation, lipoylation, attachment of flavin moiety (e.g., FMN or FAD), attachment of heme C, phosphopantetheinylation, retinylidene Schiff base formation, diphthamide formation, ethanolamine phosphoglycerol attachment, hypusine formuation, biotinylation, pegylation, ISGylation, SUMUylation, ubiquitination, Neddylation, Pupylation, citrullination, deamidation, eliminylation, carbamylation, or a combination thereof.

Compositions comprising one or more netrin-1 compounds may be subjected to one or more rounds of purification or concentration steps known in the art to remove impurities and/or concentrate the peptide fragments. Thus, in some embodiments, the present invention provides peptide compositions having a purity and/or composition not found in nature. In some cases, the peptide composition is at most 30%, 40%, 50%, 60%, 70%, 80%, 90%, 95%, 99%, 99.9%, or 100% pure peptide fragments. In some cases, the peptide composition is at least 30%, 40%, 50%, 60%, 70%, 80%, 90%, 95%, 99%, 99.9%, or 100% pure peptide fragments. In some cases, the composition is free of impurities. In some cases, the amount of the peptide fragments in the peptide composition is at most 30%, 40%, 50%, 60%, 70%, 80%, 90%, 95%, 99%, 99.9%, or 100% weight of the total composition. In some cases, the amount of the peptide fragments in the peptide composition is at least 30%, 40%, 50%, 60%, 70%, 80%, 90%, 95%, 99%, 99.9%, or 100% by weight of the total composition.

Compositions of the present invention include pharmaceutical compositions that comprise one or more netrin-1 compounds. The term “pharmaceutical composition” refers to a composition suitable for pharmaceutical use in a subject. A pharmaceutical composition generally comprises an effective amount of an active agent, e.g., one or more netrin-1 compounds according to the present invention, and a pharmaceutically acceptable carrier. The term “effective amount” refers to a dosage or amount sufficient to produce a desired result. The desired result may comprise an objective or subjective improvement in the recipient of the dosage or amount, e.g., long-term survival, effective prevention of a disease state, and the like. In some embodiments, the “effective amount” is less than a therapeutically effective amount. In some embodiments, pharmaceutical compositions comprise one or more netrin-1 compounds in a therapeutically effective amount. Pharmaceutical compositions according to the present invention may further include one or more supplementary agents. Supplementary agents include prostanoid analogues, endothelin receptor antagonists (ERAs), phosphodiesterase type 5 (PDE-5) inhibitors, and soluble guanylate cyclase (sGC) stimulators.

One or more netrin-1 compounds according to the present invention may be administered, preferably in the form of pharmaceutical compositions, to a subject. Preferably the subject is mammalian, more preferably, the subject is human. Preferred pharmaceutical compositions are those comprising at least one netrin-1 compound in a therapeutically effective amount and a pharmaceutically acceptable vehicle.

Pharmaceutical compositions of the present invention may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations of the present invention may be optimized for increased stability and efficacy using methods in the art. See, e.g., Carra et al. (2007) Vaccine 25:4149-4158.

The compositions of the present invention may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular netrin-1 compound used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy. 20th ed. (2000) Lippincott Williams & Wilkins. Baltimore, Md.

The pharmaceutical compositions of the present invention may be provided in dosage unit forms. As used herein, a “dosage unit form” refers to physically discrete units suited as unitary dosages for the subject to be treated; each unit containing a predetermined quantity of the one or more netrin-1 compounds calculated to produce the desired therapeutic effect in association with the required pharmaceutically acceptable carrier. The specification for the dosage unit forms of the invention are dictated by and directly dependent on the unique characteristics of the given netrin-1 compound and desired therapeutic effect to be achieved, and the limitations inherent in the art of compounding such an active compound for the treatment of individuals.

Toxicity and therapeutic efficacy of netrin-1 compounds and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. netrin-1 compounds which exhibit large therapeutic indices are preferred. While netrin-1 compounds that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more netrin-1 compounds according to the present invention can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Materials and Methods

### Netrin-1 Compounds

The following are exemplary netrin-1 compounds:

V1P:
(SEQ ID NO: 2)
(mini-PEG)-CDCRHNTAG
V2P:
(SEQ ID NO: 3)
(mini-PEG)-CLNCRHNTAG
V3P:
(SEQ ID NO: 4)
(mini-PEG)-CPCKDGVTIGIT
V1S:
(SEQ ID NO: 5)
SDCRHNTAG
V1T:
(SEQ ID NO: 6)
TDCRHNTAG
V1C:
(SEQ ID NO: 7)
CDCRHNTAG,
wherein the cysteine residues are joined by a
disulfide bond
V1D:
(SEQ ID NO: 8)
dCDCRHNTAdG,
wherein “d” indicates that the amino acid
residue is a D-amino acid
(SEQ ID NO: 9)
The full-length human netrin-1 protein GI
148613884
V1-9aa:
(SEQ ID NO: 10)
CDCRHNTAG
V2-10aa:
(SEQ ID NO: 11)
CLNCRHNTAG
V3-11aa:
(SEQ ID NO: 12)
CPCKDGVTGIT
V1:
(SEQ ID NO: 13)
CKCNGHAARCVRDRDDSLVCDCRHNTAGPECDRCKPFHYDRPWQRATARE
ANEC
V2:
(SEQ ID NO: 14)
CNCNLHARRCRFNMELYKLSGRKSGGVCLNCRHNTAGRHCHYCKEGYYRD
MGKPITHRKAC
V3:
(SEQ ID NO: 15)
CDCHPVGAAGKTCNQTTGQCPCKDGVTGITCNRCAKGYQQSRSPIAPC
V2-deletion:
(SEQ ID NO: 16)
NLHARRCRFNMELYKLSGRKSGGVCLNCRHNTAGRH
V3-deletion:
(SEQ ID NO: 17)
HPVGAAGKTCNQTTGQCPCKDGVTGIT

### Reagents

Mouse recombinant netrin-1 was purchased from R&D Systems (Minneapolis, Minn.). Assay kit for cholesterol, triglyceride and HDL-cholesterol were all purchased from Pointe Scientific (Canton, Mich.). Standard solutions for triglyceride and HDL-cholesterol were also purchased from Pointe Scientific. Standards for cholesterol assays were prepared using the powder obtained from Sigma-Aldrich (St Louis, Mo.). The antibody used for macrophage staining, Rat Anti-Mouse CD107b (Mac3) was purchased from BD Biosciences (San Jose, Calif.). M1 macrophage marker IL-1β Mouse monoclonal antibody and M2 macrophage marker Arginase-1 Rabbit monoclonal antibody were purchased from Cell Signaling Technology (Danvers, Mass.). 4′6-Diamidino-2-Phenylindole, Dihydrochloride (DAPI) solution was from Invitrogen (Carlsbad, Calif.). Histopaque 1083 (Sigma-Aldrich, St Louis, Mo.) and calcein AM (Calbiochem, Germany) were prepared for monocyte isolation and labeling. Two different antibodies for UNC5B were purchased from Abcam (Cambridge, Mass.) and Enzo Life Sciences (Farmingdale, N.Y.).

### Animals

All animal procedures were approved by the Institutional Animal Care and Usage Committee at the University of California, Los Angeles (UCLA). Breeders of apoE^{-/-} mice were purchased from Jackson Laboratory (Bar Harbor, Me., Strain B6.129P2-Apoe^{tmlUnc}/J) and bred in house till experimental use. Twelve- to fourteen-weeks old male animals were infused with or without netrin-1 (15 ng/day) a day in advance to the commencement of high fat diet (HFD/42% fat; Harlan Laboratories, Madison, Wis.) for 16 weeks.

### Netrin-1 Compound Infusion Using Osmotic Pumps

Netrin-1 compounds were delivered by infusion using osmotic pumps. In order to complete 16 weeks treatment, osmotic pumps were replaced twice during the HFD. Two 6-week pumps and a 4-week pump were used (ALZET, Cupertino, Calif.) per animal. Animals were anesthetized with isoflurane in a closed chamber and moved onto surgical stage where an inhalation anesthesia mask is provided. While supplying 95%/5% of O₂/CO₂ mixed with 1.5% isoflurane, hair at lower posterior neck area was removed and disinfected using ethanol and iodine solution. A small incision was made at this area and 6-week or 4-week osmotic pump was inserted subcutaneously.

### Tissue Collection

After 16-week of HFD feeding, animals were weighed and sacrificed by euthanizing with CO₂. Heparin (0.1 mL) was injected via left ventricle and left it to circulate for a minute and blood was collected slowly from right ventricle using 14G needle. The collected blood was used to analyze lipid profile. The animals were then perfused with ice-cold PBS to flush out remaining blood in the body. The entire lobes of liver was removed to record its weight for each animal. The aorta connecting the heart was excised immediately from the body and the connective tissue was cleaned under a surgical microscope. The upper quarter of the heart including the root of aorta was used for histological analysis because tricuspid/aortic root tends to show atherosclerotic lesion most frequently compare to the other part of aorta. Rest of the part of aorta including ascending aorta, aortic arch, descending aorta and iliac artery branch point, was harvested and opened en face for Oil Red-O staining.

### Plasma Lipid Profile Measurement

The blood collected from right ventricle was placed in a 1.5 mL centrifuge tube and centrifuged at 2000 rpm for 10 minutes. The semi-transparent plasma phase was replaced into a new centrifuge tube and used for each assay. Levels of cholesterol, triglyceride and HDL-cholesterol were measured based on colorimetric change per manufacturer's instruction. The absorbance at 500 nm was detected using Bio-Tek plate reader.

### Oil Red-O Staining on Aorta Dissected En Face

Three percent of oil red-O solution was prepared in 2-propanol and the reagent was heated to 56° C. for 1 hour. The oil red-O reagent was diluted to 1.8% by mixing with water and filtered with a 0.22 μm syringe filter. Aorta opened en face was rinsed once with 0.5% triton including PBS (PBST) and then stained with working oil red-O solution for 30 minutes at room temperature. Following the staining, aorta was washed with 2-propanol for a min and returned to PBST for additional wash. The aorta image was obtained under the Nikon E600 microscope and the stained lesion area was calculated using ImageJ.

### Aortic Root Histological Analysis

The upper quarter of heart including aortic root area was sliced and collected into 4% of paraformaldehyde for 0/N for fixation. Tissues were displaced in 10% sucrose for at least a few hour prior to being embedded in paraffin for sectioning at 5 μm. The paraffin section slicing and H&E staining was performed using the standard protocols in the Translational Pathology Core Laboratory Core Facility at UCLA. The slices tissue sections were also used for macrophage staining using anti-CD107b (Mac 3) antibody. All staining was performed double-blindly.

### Monocyte Isolation

Monocytes were isolated from the bone marrow of 6 to 8-week old male C57BL/6 mice. Femur and tibia bones were harvested and cleaned, and ice-cold PBS was flushed into the bones using insulin syringe. Histopaque 1083 was used to separate mononuclear cells by centrifuging at 400×g for 30 minutes at room temperature. The middle opaque layer was carefully replaced and washed with RPMI1640.

### Monocyte Endothelial Adhesion Assay

Bovine aortic endothelial cells (BAECs) were cultured in M199 medium on a 96-well plate until more than 80% confluent. On the day of the assay, 100 ng/mL of TNFα was added to BAECs while harvesting monocytes from the bone marrow. The concentration of 1.0×10⁶ cells/mL of isolated monocytes were labeled with calcein AM. Meanwhile, BAECs were treated with or without PTIO for 30 minutes. After 30 minutes of labeling, the monocytes were washed twice with medium and treated with or without anti-UNC5B antibody (1 μg) to block UNC5B receptor from binding with netrin-1. At the same time, netrin-1 (100 ng/mL) was added to BAECs. Both cells underwent another 30 minutes of incubation. At the end of the incubation, the medium of BAECs were removed and washed with warm PBS once. Then monocytes in 100 μL PBS were overlaid on top of BAECs and co-cultured for 30 minutes. Cells were gently washed with PBS twice to remove unattached cells. The level of calcein AM labeled monocytes adhere to BAECs were detected by reading at excitation/emission: 485/528 nm in a Bio-Tek fluorescence plate reader.

### UNC5B Detection in Monocytes

In order to observe the cleavage of UNC5B in response to netrin-1 binding, monocytes were treated with or without netrin-1 for 30 minutes. After the treatment, the pellet of cells were lysed, and subjected to detection of intact UNC5B and cleaved UNC5B protein levels by Western blotting per standard protocols using 7.5% SDS/PAGE and nitrocellulose membranes. Antibody from Abcam (#ab139643/1:500 dilution) was used to detect intact form of UNC5B, whereas the antibody from Enzo Life Sciences (#ALX-804-846-C100/1:500 dilution) was applied for detection of cleaved-UNC5B. The ratio of expression levels of cleaved-/intact-UNC5B was calculated.

### Additional Experiments and Embodiments

### Potentiation of Netrin-1 Activity

To determine the effects of netrin-1 compounds on monocyte activation and its dependency on UNC5B; whether inhibition of p47phox potentiates abrogation of restenosis and atherosclerosis via inhibition of UNC5B by netrin-1 compounds; and the effects of netrin-1 compounds on vascular smooth muscle cell (VSMC) proliferation and migration and macrophage infiltration, and the underlying molecular mechanisms, the following experiments may be conducted.

Monocyte recruitment into injury site is a major component facilitating restenosis and atherosclerosis. Overexpression of monocyte chemoattractant protein (MCP)-1/CCL2 for chemokine (C—C motif) ligand 2 induces macrophage infiltration and atherosclerotic lesion formation, while MCP-1 deficiency or inhibition is associated with reduced post injury intimal hyperplasia. MCP-1 is responsible for recruiting monocytes, which later become macrophages within the vessel wall.

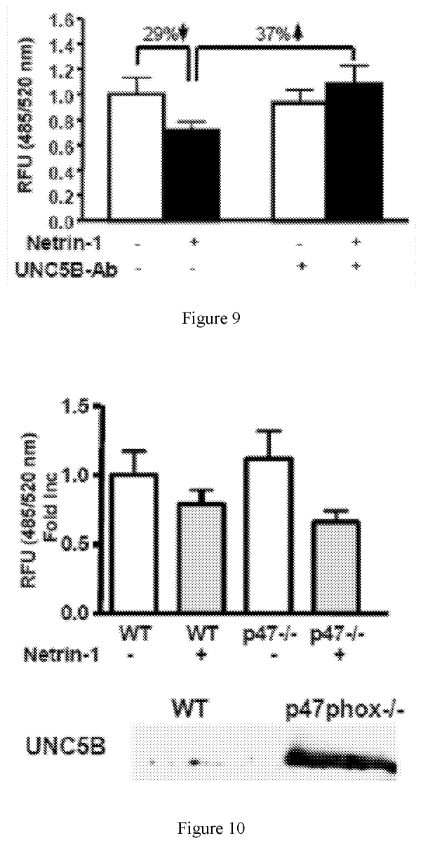

In preliminary experiments, it was found that netrin-1 attenuates monocyte migration in a UNC5B dependent manner (FIG. 8, Panel B, FIG. 9, FIG. 10). In additional preliminary studies, MCP-1 expression was found upregulated after femoral artery wire injury, but attenuated by netrin-1 infusion. UNC5B is uniquely expressed in monocytes and absent in endothelial cells and cardiomyocytes.

Monocyte migration and adhesion to endothelial cells may be determined in the presence or absence of netrin-1 compound treatment and UNC5B antibody to elucidate effects of netrin-1 on monocyte activation, as well as the dependency on UNC5B of these regulations. Monocyte activation and MCP-1 expression in situ in injured femoral arteries and aortas of high-fat fed apoE null mice and LDL receptor (LDLR) deficient mice with and without netrin-1 compound infusion and injection of endothelial progenitor cells (EPCs) pre-conditioned with netrin-1 compounds may also be examined. Besides inflammation, VSMC proliferation and migration play important roles in restenosis and atherosclerosis. Following the initial endothelial damage, loss of NO results in loss of NO-dependent inhibition of VSMC proliferation and migration. Therefore, it is hypothesized that by generating NO, netrin-1 compounds attenuate restenosis and atherosclerosis via inhibition of VSMC proliferation and migration.

Monocytes are isolated from bone marrow of 6-8 weeks old C57BL6 mice. For analysis of regulation of monocyte migration by netrin-1 compounds, cells are seeded on transwell chamber in serum free medium, in the presence or absence of neutralizing UNC5B antibody (2 μg/ml). The transwell is placed on a 24-well plate filled with 5% FBS containing RPMI 1640 medium, with and without netrin-1 compounds (100 ng/ml). After 4 hours of incubation, cells on the bottom surface of transwell, which are the migrated population, are treated with dissociation buffer (Trevigen) containing calcein AM (Calbiochem). The fluorescence of calcein AM intensity is measured using Bio-Tek fluorescent plate reader at excitation and emission of 485 nm and 520 nm, respectively (Synergy HT, Bio-Tek).

In preliminary experiments, netrin-1 inhibition of monocyte migration was found to be abrogated by UNC5B antibody (FIG. 9). Monocyte adhesion to endothelial cells may be analyzed using methods in the art. After treatment with netrin-1 (100 ng/ml, 12 hours) in the presence or absence of UNC5B antibody (2 μg/mL), monocytes are labeled with Vybrant DiI or DiO solution (Molecular Probes) in serum-free RPMI1640 medium. The labeled monocytes are added (2×10⁵ cells/well) to confluent monolayer of bovine aortic endothelial cells cultured in 24-well plates. After incubation for at 37° C. non-adherent cells are removed by washing with RPMI. The fluorescent-labeled monocytes are counted under fluorescent microscope.

Additional preliminary data indicated an intriguing regulatory effect of p47phox on UNC5B expression. Knockout of p47phox resulted in marked upregulation of UNC5B indicating that p47phox is suppressive of UNC5B expression (FIG. 10). Due to increased UNC5B abundance, netrin-1 was more effective in attenuating monocyte migration in p47phox knockout mice (FIG. 10). Netrin-1 increased levels of cleaved UNC5B, which might make it less available to guide monocyte migration, resulting in diminished migrating activity (FIG. 8, Panel B).

p47phox/apoE DKO mice may be used to examine whether any reduction in atherosclerosis was derived from reduced monocyte activation, by in situ quantitation of monocyte infiltration, and to examine responses in monocyte activation in vivo in relation to UNC5B and restenosis as follows.

Activation of monocytes in situ may be assessed by monocyte infiltration into the wound site using a fluorescent probe of MOMA-2 (monoclonal anti-mouse) (MC and MO marker) and quantified, on Day 1 and Day 7 post femoral artery injury, in femoral artery injured mice with and without netrin-1 compound infusion (15 ng/kg/day in osmotic minipumps) and injection of EPCs (500 cells) pre-conditioned with netrin-1 compounds. It is hypothesized that pre-conditioned EPCs will also inhibit monocyte activation. The femoral artery wire injury model for neointimal formation/restenosis known in the art is used. MCP-1 expression in situ is evaluated by immunohistochemistry at 1 and 4 weeks post femoral artery injury (antibody from Cell Signaling, 1:50). All in vivo experiments contain 6 groups of sham, sham/netrin-1, sham/netrin-1-EPC, injury, injury/netrin-1, injury/netrin-1-EPC, using both neointima/restenosis model (femoral artery injury) and atherosclerosis model (high fat diet fed apoE null and LDLR deficient mice). Additional groups may include p47phox knockout mice and p47phox/apoE DKO mice.

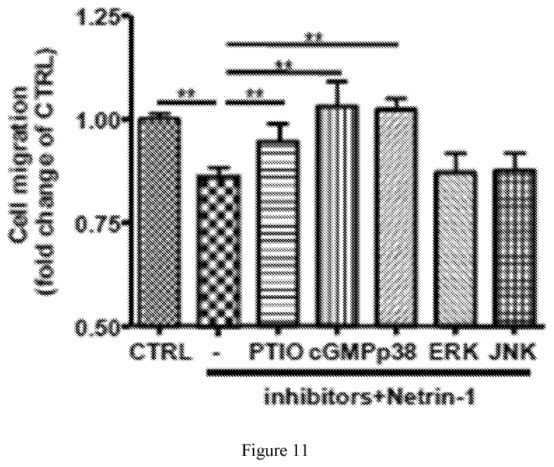

Proliferation and migration of VSMC is a hallmark of restenosis and atherosclerosis. It is hypothesized that netrin-1 attenuation of restenosis is at least partially attributed to inhibition of VSMC proliferation and migration. In preliminary experiments, VSMC migration was abrogated by netrin-1 treatment, which was reversed by scavenging NO, antagonization of cGMP, and inhibition of p38 MAPK (FIG. 11, EC-VSMC co-culture system). ERK1/2 and JNK however seemed not involved. In addition, p38 MAPK phosphorylation in response to netrin-1 was inhibited by cGMP antagonist. Therefore, the roles of different p38 MAPK isoforms may be delineated. Four different p38 isoforms exist including the prototypic p38a (often as p38), p38β, p38γ, and p38δ. p38 and p38β are ubiquitously expressed, while p38γ is expressed primarily in skeletal muscle and p38δ is found in lung, kidney, testis, pancreas, and small intestine. Therefore, potential differential roles of p38/p38β in mediating netrin-1 inhibition of VSMC migration may be examined.

To examine whether netrin-1 regulation of VSMC migration is mediated specifically by endothelial cell production of NO, a new system of EC-VSMC co-culture system is adapted to examine VSMC migration using a wound assay. The endothelial cells are separately cultured on transwell inserts and exposed to netrin-1 compounds, prior to being overlaid on top of the VSMC cells (RASMCs, Lonza) grown on a 6-well plate with wound created at baseline and followed overtime to examine responses after netrin-1 stimulated endothelial cell inserts are placed on top. Pharmacological inhibitors of U0126 (ERK1/2, 50 μmol/L), SB202190 (p38, 10 μmon), SP600125 (JNK, 10 μmon), Rp-8-Br-PET-cGMPs (cGMP, 10 μmon), and PTIO (NO scavenger, 60 μmon) are added to VSMC culture before endothelial cell inserts are placed on top. The migrating/wound closure activity of VSMC monolayer is followed and pictures taken for analyses using Image J software.

VSMC proliferation is determined using an MTT assay known in the art. Following 24, 48, 72 or 96 hours of incubation of VSMC with netrin-1 (100 ng/ml); 20 IA of 5 mg/mL MTT is added to the 96 well plates and incubated for 3 hours. At the end of the incubation, the purple formazan crystals are dissolved with the addition of 150 μL DMSO and absorbance determined at 540 nm wavelength using Bio-Tek fluorescent plate reader. For analysis of VSMC proliferation in situ, PCNA and Ki67 staining is carried out as described for restenosis models for PCNA. Macrophage infiltration in situ is examined by Mac-3 staining, and in preliminary experiments, marked infiltration of macrophages in situ in high fat diet fed apoE null mice were substantially attenuated by netrin-1 infusion (data not shown).

Anticipated Results and Data Interpretation. Netrin-1 compounds are expected to consistently inhibit monocyte activation including migration and adhesion to endothelial cells, MCP-1 expression in situ in injured femoral arteries and aortas of high-fat fed apoE null mice/LDLR deficient mice. Changes in migration are expected to be due to activation of UNC5B by netrin-1 compounds, as the inhibitory effects would be lost in cells treated with UNC5B neutralizing antibody or in aortas of UNC5B knockout mice. Redox sensitive regulatory mechanisms of UNC5B expression based on data from p47phox deficient mice are expected to indicate that inhibition of monocyte activation by netrin-1 compounds occurs by upregulation of UNC5B on monocytes. Netrin-1 compounds are expected to consistently inhibit VSMC migration via the NO/cGMP/PKG/p38 MAPK pathway.

### Attenuation of UNC5B-Dependent Monocyte Activation

To determine whether administration of netrin-1 compounds or EPCs pre-conditioned with netrin-1 compounds attenuates dyslipidemia and atherosclerosis in high-fat fed apoE null and LDLR deficient mice, the underlying molecular mechanisms involving attenuation of monocyte and VSMC activation and attenuation of dyslipidemia by netrin-1 compounds may be examined.

Augmented EPC function and attenuated VSMC migration mediate netrin-1 prevention of restenosis after femoral artery wire injury of endothelium. These effects are mediated by DCC receptor dependent preservation of EPC survival and NO-mediated inhibition of VSMC migration. The unique role of p47phox in mediating UNC5B cleavage to regulate UNC5B-dependent monocyte responses and the inhibitory effect on VSMC migration and mechanisms thereof by netrin-1 compounds may be examined using an EC-VSMC co-culture in which endothelial cells are exposed to netrin-1 compounds before being layered (on inserts) on top of a wounded VSMC monolayer. The migratory activity of VSMCs to close the wound may be imaged and quantified. NO scavenger PTIO and other signaling pathway inhibitors may be used to pretreat VSMCs to reveal the downstream signaling and mechanisms related to endothelial production of NO. Based on the effects of netrin-1 on regulating EPCs, monocytes and VSMCs that all play major roles in atherosclerosis and restenosis, high fat diet fed apoE null mice and LDLR deficient mice may be used to examine effects of netrin-1 on atherosclerosis and dyslipidemia as follows.

To examine effects of netrin-1 on atherosclerosis, 12-14 weeks wildtype and apoE null or LDLR deficient mice are fed with high fat diet (42% fat, Harlan Labs) with or without being infused with netrin-1 compounds in osmotic minipumps (15 ng/day) for 16 weeks. As shown in FIG. 1 and FIG. 2, netrin-1 was robustly protective in fully attenuating fatty liver, lesion formation, and dyslipidemia of hypercholesterolemia and hypertriglyceridemia in apoE null mice. These experiments and similar treatment with netrin-1 compounds in LDLR deficient animals may be conducted as follows. On the day of harvest, body weight and organ weights are measured. Plasma lipid levels including LDL-cholesterol are determined. In brief, plasma is freshly prepared and total cholesterol are measured using a cholesterol reagent colorimetric assay kit (Pointe Scientific, #C7510). For HDL cholesterol measurement, plasma is incubated with a precipitating reagent (Pointe Scientific, #H7511) to remove LDL and VLDL before the colorimetric assay. LDL cholesterol levels are determined using autoLDL Cholesterol Reagent kit (Pointe Scientific, #7574). Non-LDL lipoprotein particles are separated and consumed by Reagent 1 provided in the kit. Then LDL cholesterol levels are determined using Reagent 2 provided in the kit. Triglyceride levels are measured calorimetrically using the triglyceride reagent kit (Pointe Scientific, #T7532). Upon harvest, the full length of aortas are isolated freshly and subjected to Oil Red staining for assessment of atherosclerotic lesions. Infiltration macrophages and lipid-laden CD36+ macrophages/foam cells are quantified by Mac-3 staining and CD36 staining.

To examine whether UNC5B-dependent monocyte activation is attenuated by netrin-1 to contribute to retarded atherogenesis, UNC5B knockout mice may be crossed with apoE null mice, and then subjected to high fat diet feeding prior to analyses of monocyte activation in situ and analyses of lesion formation. To determine if netrin-1 attenuation of VSMC activation is involved in reduced atherogenesis, markers of VSMC activation in situ may be examined. PKG inhibitors may be given to the mice to examine if disruption of the inhibitory effects on VSMC activation will blunt the protective effects of netrin-1 on atherosclerosis. Preliminary data indicates that netrin-1 remarkably treats dyslipidemia. Since netrin-1 primarily targets endothelial cells in the blood vessels and the heart, netrin-1 compounds may be used to lower lipids in subjects and thereby treat diseases and disorders related to dyslipidemia such as atherosclerosis.

To compare the effects of netrin-1 compounds with existing pathways to lower cholesterol, whether netrin-1 compounds inhibit HMG-CoA reductase and PCSK9 activity may be examined as follows. For the biosynthetic pathway, the HMG-CoA reductase activity in the liver is assayed using a kit from Sigma. The phosphorylation of the enzyme, and its degradation as well as mRNA expression levels, are examined to elucidate detailed regulatory mechanisms by netrin-1 compounds. PCSK9 is the hepatic protease that attaches and internalizes LDL receptors into lysosomes hence promoting their destruction. The PCSK9 activity is determined assayed using a kit from Abcam in the apoE null mice. The effects of netrin-1 compounds on circulating levels of oxLDL and LDLR expression may also be analyzed in the apoE null mice.

In parallel experiments, identically fed apoE null or LDLR deficient mice are injected with EPCs pre-conditioned with netrin-1 compounds, which pre-conditioned EPCs are anticipated to produce NO that thereby inhibits various aspects of atherosclerosis including monocyte and VSMC activation. EPCs are prepared using methods in the art and then preconditioned with netrin-1 (500 cells, 100 ng/ml netrin-1 compounds) ex vivo prior to being injected to the mice every 24 hours for the entire study period of 16 weeks. Animals are then examined for evidence and markers of atherosclerosis.

Anticipated Results and Data Interpretation. Netrin-1 compounds exhibit a robust inhibitory effect on high fat feeding induced atherosclerosis and dyslipidemia in apoE null mice and comparable results are expected in LDLR deficient mice. Netrin-1 compounds are expected to augment EPC function and attenuate monocyte and VSMC activation.

### Upregulation of Endogenous Netrin-1 Signaling

To determine whether endogenous netrin-1 signaling is physiologically protective against restenosis and atherosclerosis, loss of which exaggerates vascular pathologies and to determine whether amplification of the endogenous signaling with exogenous administration of netrin-1 is necessary to achieve sufficient protection, the following experiments may be conducted.

Exogenously applied netrin-1 compounds are robustly cardioprotective and protective of restenosis. The protection against restenosis and atherosclerosis are hypothesized to be mediated by augmented EPC function to increase re-endothelialization, abrogated VSMC proliferation and migration, as well as attenuated monocyte activation in the vessel wall. In the heart, NO production from netrin-1 attenuates activation of NADPH oxidase isoform 4 to diminish oxidative stress, eNOS uncoupling, mitochondrial dysfunction, and chronically autophagy and cardiac remodeling post-myocardial infarction. Netrin-1 and its receptor DCC (mediating NO production from ECs and EPCs) are also endogenously expressed in endothelial cells and cardiomyocytes under physiological conditions. The data herein indicates that endogenous netrin-1 signaling is important for physiological preservation of NO-dependent cardioprotection, loss of which results in worsened cardiac injury post ischemia reperfusion insult. The endogenous role of netrin-1 compounds in vascular protection may be examined using netrin-1 knockout mice for the restenosis/neointimal model, and netrin-1/apoE double knockout mice for the analysis of atherosclerosis. Molecular mechanisms underlying physiological vascular protection by netrin-1 compounds may also be examined likely similarly involving NO production, enhanced EPC homing, and attenuated in situ monocyte and VSMC activation as described above. It is hypothesized that amplification of endogenous signaling with exogenous supplementation is necessary for sufficient protection against vascular pathologies.

To examine an endogenous role of netrin-1 in protecting against restenosis, netrin-1 knockout mice are exposed to femoral artery injury model and harvested for analysis of neointimal formation. For these experiments, 8-10 weeks old male and female WT and netrin-1 deficient mice are subjected to femoral artery wire injury and harvested 4 weeks later, using the non-injured side of the femorals as controls. For the femoral artery injury protocol, an incision is made on the right thigh area above the femoral artery. The femoral artery is isolated and cleared of connective tissue. Blood flow is temporarily stopped using a small artery clamp, and an incision is made on a branch between the rectus femoris and vastus medialis muscles. A wire guide coated with heparin is inserted into the artery and moved up. The artery clamp is then removed and the wire moved up toward the iliac artery. The wire is allowed to stay for 60 seconds, and pulled back towards the incision point; this motion repeated 3 times. Then the artery is once again clamped and the wire guide pulled out of the artery. The incision site on the artery is then be ligated using surgical silk (5-0 Vicryl). The skin was closed and sutured, and sealed with surgical glue. The injury surgery was performed on the right leg, while left femoral artery was left intact to serve as controls.

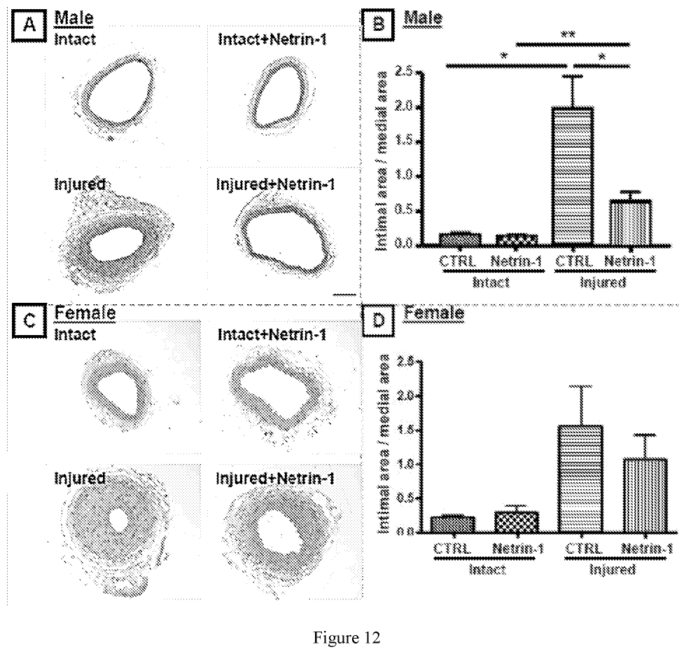

For histology analyses, animals are perfused with 4% paraformaldehyde after being euthanized. Then tissues are harvested at the appropriate time points and embedded in paraffin (e.g., samples taken at 7 days and 28 days for vascular morphology) and OCT (e.g., samples taken at 1 hour and 24 hours for immunofluorescent staining of in situ analyses of EPC homing and activation of monocyte and VMSC), and subjected to slicing at 5 μm. The intimal growth is evaluated by analyzing areas of the intimal and medial layers from the H&E staining, at Day 7 or Day 28 post-injury. For acute response, CD34 and isolectin GS-IB4 antibodies are used to detect homing EPCs and residential endothelial cells, on 1-hour post-injury tissues. Further, PCNA staining is performed using VECTASTATIN Kit (Vector Labs). Ki67 is detected using fluorescent ab, and contrasted with isolectin to differentiate regulations in VSMC vs. endothelial cells. Images are quantified using Image J software. Netrin-1 infusion (15 ng/day, 4 weeks) into injured mice abolished neointimal formation and restenosis in male and female mice (FIG. 12, Panels A-D). These data establish rigorous analyses in both genders of animals, and feasibilities of all experimental protocols to assess neointimal growth/restenosis including quantification.

Worsened restenosis/neointimal formation is expected for femoral artery injured netrin-1 knockout mice. In further experiments, netrin-1/apoE double knockout mice are generated and subjected to high fat feeding to induce atherosclerosis, and deteriorated atherosclerosis as defined by oil red staining is expected. Homing of EPCs, and in situ activation of monocytes and VSMC may also be examined in these mice as described above. Plasma lipid profiles are assessed. In some experiments, the netrin-1/apoE double knockout mice are infused with different dosage of netrin-1 (5, 15 ng/day, 16 weeks) to examine if this is sufficient to attenuate lesion formation to the level of apoE null mice with low dose and to the control level with high dose that was effective in substantially reducing lesion formation.

Anticipated Results and Data Interpretation. Netrin-1 deficiency associated with deteriorated restenosis/neointimal formation in femoral artery wire injured netrin-1 knockout mice of both genders is expected. This response is expected to be associated with worse EPC function characterized by reduced homing, and activation of monocyte and VSMC in situ in the wound site. High fat feeding into netrin-1/apoE double knockout mice expected to lead to worse lesion formation, accompanied by worse dyslipidemia and body weight gain/development of fatty liver, compared to apoE null mice alone. However, netrin-1 infusion into the DKO mice is expected to result in partial or full attenuation of lesion formation and related phenotypes to apoE null or control animal levels. Taken together, these data would indicate that amplification of netrin-1 signaling, by either increasing endogenous expression of netrin-1 or administration of exogenous netrin-1 compounds, is important for vascular protection.

### REFERENCES

The following references are herein incorporated by reference in their entirety with the exception that, should the scope and meaning of a term conflict with a definition explicitly set forth herein, the definition explicitly set forth herein controls:
[1] J. Jawien, “The Role of An Experimental Model of Atherosclerosis: apoE-knockout Mice in Developing New Drugs against Atherogenesis,” Curr. Pharm. Biotechnol., vol. 13, no. 13, pp. 2435-2439, 2012.
[2] M. F. Linton, P. G. Yancey, S. S. Davies, W. G. (Jay) Jerome, E. F. Linton, and K. C. Vickers, The Role of Lipids and Lipoproteins in Atherosclerosis, vol. 111, no. 2877. 2000.
[3] W. Insull, “The Pathology of Atherosclerosis: Plaque Development and Plaque Responses to Medical Treatment,” Am. J. Med., vol. 122, no. 1 SUPPL., 2009.
[4] D. Gradinaru, C. Borsa, C. Ionescu, and G. I. Prada, “Oxidized LDL and NO synthesis-Biomarkers of endothelial dysfunction and ageing,” Mechanisms of Ageing and Development, vol. 151. pp. 101-113, 2015.
[5] T. Heitzer et al.,“Tetrahydrobiopterin improves endothelium-dependent vasodilation in chronic smokers: evidence for a dysfunctional nitric oxide synthase.,” Circ Res, vol. 86, no. 2, pp. E36-E41, 2000.
[6] C. Muller, R. Salvayre, A. Négre-Salvayre, and C. Vindis, “Oxidized LDLs trigger endoplasmic reticulum stress and autophagy: Prevention by HDLs,” Autophagy, vol. 7, no. 5. pp. 541-543, 2011.
[7] A. Nguyen and H. Cai, “Netrin-1 induces angiogenesis via a DCC-dependent ERK1/2-eNOS feed-forward mechanism.,” Proc. Natl. Acad. Sci. U.S.A, vol. 103, no. 17, pp. 6530-5, 2006.
[8] N. M. Liu, K. L. Siu, J. Y. Youn, and H. Cai, “Attenuation of neointimal formation with netrin-1 and netrin-1 preconditioned endothelial progenitor cells,” J. Mol. Med., vol. 95, no. 3, pp. 335-348, 2017.
[9] D. Salisbury and U. Bronas, “Inflammation and immune system contribution to the etiology of atherosclerosis: Mechanisms and methods of assessment,” Nursing Research, vol. 63, no. 5. pp. 375-385, 2014.
[10] D. A. Chistiakov, A. V. Grechko, V. A. Myasoedova, A. A. Melnichenko, and A. N. Orekhov, “The role of monocytosis and neutrophilia in atherosclerosis,” Journal of Cellular and Molecular Medicine, vol. 22, no. 3. pp. 1366-1382, 2018.
[11] N. P. Ly et al., “Netrin-1 inhibits leukocyte migration in vitro and in vivo,” Proc Natl Acad Sci USA, vol. 102, no. 41, pp. 14729-14734, 2005.
[12] N. A. Elshourbagy, H. V. Meyers, and S. S. Abdel-Meguid, “Cholesterol: The good, the bad, and the ugly-therapeutic targets for the treatment of dyslipidemia,” Medical Principles and Practice, vol. 23, no. 2. pp. 99-111, 2014.
[13] Y. Li, T. R. Gilbert, A. H. Matsumoto, and W. Shi, “Effect of aging on fatty streak formation in a diet-induced mouse model of atherosclerosis,” J. Vasc. Res., vol. 45, no. 3, pp. 205-210, 2008.
[14] H. Arakawa, “p53, apoptosis and axon-guidance molecules,” Cell Death and Differentiation, vol. 12, no. 8. pp. 1057-1065, 2005.
[15] U. Landmesser, B. Hornig, and H. Drexler, “Endothelial dysfunction in hypercholesterolemia: mechanisms, pathophysiological importance, and therapeutic interventions.,” Semin. Thromb. Hemost., vol. 26, no. 5, pp. 529-37, 2000.
[16] Y. Baumer et al., “Hyperlipidemia-induced cholesterol crystal production by endothelial cells promotes atherogenesis,” Nat. Commun., vol. 8, no. 1, 2017.
[17] S. Bandeali and J. Farmer, “High-density lipoprotein and atherosclerosis: The role of antioxidant activity,” Curr. Atheroscler. Rep., vol. 14, no. 2, pp. 101-107, 2012.
[18] G. F. Lewis and D. J. Rader, “New insights into the regulation of HDL metabolism and reverse cholesterol transport,” Circulation Research, vol. 96, no. 12. pp. 1221-1232, 2005.

All scientific and technical terms used in this application have meanings commonly used in the art unless otherwise specified.

Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide”, “peptide”, and “peptide fragments” are used interchangeably to refer to two or more natural and/or unnatural amino acids linked together and one letter amino acid designations are used in the sequences and formulas herein. As used herein, “aa” is an abbreviation used for “amino acids”. For example, the “9aa” of “V1-9aa” indicates that the peptide is 9 amino acid residues long.

As used herein, a given percentage of “sequence identity” refers to the percentage of nucleotides or amino acid residues that are the same between sequences, when compared and optimally aligned for maximum correspondence over a given comparison window, as measured by visual inspection or by a sequence comparison algorithm in the art, such as the BLAST algorithm, which is described in Altschul et al., J. Mol. Biol. 215:403-410 (1990). Software for performing BLAST (e.g., BLASTP and BLASTN) analyses is publicly available through the National Center for Biotechnology Information (ncbi.nlm.nih.gov). The comparison window can exist over a given portion, e.g., a functional domain, or an arbitrarily selection a given number of contiguous nucleotides or amino acid residues of one or both sequences. Alternatively, the comparison window can exist over the full length of the sequences being compared. For purposes herein, where a given comparison window (e.g., over 80% of the given sequence) is not provided, the recited sequence identity is over 100% of the given sequence. Additionally, for the percentages of sequence identity of the proteins provided herein, the percentages are determined using BLASTP 2.8.0+, scoring matrix BLOSUM62, and the default parameters available at blast.ncbi.nlm.nih.gov/Blast.cgi. See also Altschul, et al. (1997), Nucleic Acids Res. 25:3389-3402; and Altschul, et al. (2005) FEBS J. 272:5101-5109.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method of treating, reducing, or inhibiting a hyperlipidemia condition in a subject, which comprises administering to the subject one or more netrin-1 compounds, wherein the one or more netrin-1 compound comprise, consist essentially of, or consist of SEQ ID NO: 1 as follows:

| | |
|---|---|
| X1-X2-X3-C-X4-X5-X6-X7-T-X8-G | (SEQ ID NO: 1) |

wherein
X1 is Ala, Asn, Cys, D-Cys, Ser, or Thr, preferably X1 is Cys, D-Cys, Ser, or Thr, and wherein X1 may be linked to the cysteine at the fourth amino acid position or an ethylene oxide compound;
X2 is present or absent, and if present, X2 is Ala, Asp, Ile, Leu, Met, Phe, Pro, Trp, or Val, preferably X2 is Leu or Pro;
X3 is present or absent, and if present, X3 is Asn, Arg, Asp, Cys, Gln, Glu, Gly, Ser, Thr, or Tyr, preferably X3 is Asn or Asp;
X4 is Arg, His, or Lys, preferably X4 is Arg or Lys;
X5 is Arg, Asp, Glu, His, Lys, Phe, Trp, or Tyr, preferably X5 is Asn, Asp, or His;
X6 is Asn, Cys, Gln, Gly, Ser, Thr, Tyr, or Val, preferably X6 is Asn or Gly;
X7 is present or absent, and if present, X7 is Asn, Gly, His, Ile, Thr, or Val, preferably X7 is Val; and
X8 is present or absent, and if present, X8 is Ala, Asn, Ile, Leu, Met, Phe, Pro, Thr, Trp, or Val, preferably X8 is Ala; and
wherein X2, X3, or both X2 and X3 are present; and
wherein one or both the amino acid residues at the 10th and 11th amino acid positions may be D-amino acids.

2. The method according to claim 1, wherein the ethylene oxide compound is polyethylene glycol (PEG), polyethylene oxide (PEO), and polyoxyethylene (POE), methoxypolyethylene glycol (MPEG), or monomethoxypolyethylene glycol (mPEG), or diethylene glycol (mini-PEG), preferably the ethylene oxide compound is mini-PEG.

3. The method according to claim 1, wherein the netrin-1 compound is a peptide having an amino acid sequence that comprises, consists essentially of, or consists of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

4. The method according to any one of claims 1-3, wherein the netrin-1 compound is about 8-60, about 8-55, about 8-50, about 8-45, about 8-40, about 8-35, about 8-30, about 8-25, about 8-20, about 8-15, about 8-12, 8-11, about 9-60, about 9-55, about 9-50, about 9-45, about 9-40, about 9-35, about 9-30, about 9-25, about 9-20, about 9-15, about 9-12, or 9-11 amino acid residues long, preferably the netrin-1 compound is 8, 9, 10, or 11 amino acid residues long.

5. The method according to claim 1, wherein the netrin-1 compound is a peptide that comprises, consists essentially of, or consists of an amino acid sequence that has at least 90% sequence identity to SEQ ID NO: 9.

6. The method according to any one of claims 1-5, wherein the one or more netrin-1 compounds are administered in the form of a pharmaceutical composition.

7. The method according to any one of claims 1-6, wherein the hyperlipidemia condition is selected from the group consisting of hyperlipidemia, hypercholesterolemia, obesity, fatty liver, fat deposits in arteries, arterial macrophage infiltration, atherosclerotic lesions, monocyte migration, vascular smooth muscle cell migration, monocyte adhesion to endothelial cells, neointimal formation, and restenosis.

8. The method according to any one of claims 1-6, wherein the hyperlipidemia condition is selected from the group consisting of hyperlipidemia, hypercholesterolemia, obesity, fatty liver, fat deposits in arteries, arterial macrophage infiltration, atherosclerotic lesions, monocyte migration, vascular smooth muscle cell migration, and monocyte adhesion to endothelial cells.

9. The method according to any one of claims 1-6, wherein the administration of the one or more netrin-1 compounds results in the subject having a lower total body fat content as compared to a negative control or as compared to the subject's total body fat content before administration of the one or more netrin-1 compounds.

10. The method according to any one of claims 1-6, wherein the administration of the one or more netrin-1 compounds results in the subject having a lower total body weight as compared to a negative control or as compared to the subject's total body weight before administration of the one or more netrin-1 compounds.

11. The method according to any one of claims 1-6, wherein the administration of the one or more netrin-1 compounds results in the subject having a lower cholesterol level as compared to a negative control or as compared to the subject's cholesterol level before administration of the one or more netrin-1 compounds.

12. The method according to any one of claims 1-6, wherein the administration of the one or more netrin-1 compounds results in the subject having a lower low density lipid (LDL) level as compared to a negative control or as compared to the subject's LDL level before administration of the one or more netrin-1 compounds.

13. The method according to any one of claims 1-12, wherein the hyperlipidemia condition is the result of a high-fat diet.


SEQUENCE LISTING (17 sequences)

SEQ ID NO: 1 - protein, 11 aa, Artificial Sequence
  Peptide based on human Netrin-1 sequence
  MISC_FEATURE (1)..(1): Xaa is Ala, Asn, Cys, Ser, or Thr, and wherein when X1 is Cys, it is linked to either the cysteine residue at the fourth amino acid position via a disulfide bond or an ethylene oxide
  MISC_FEATURE (2)..(2): Xaa is present or absent, and if present, Xaa is Ala, Asp, Ile, Leu, Met, Phe, Pro, Trp, or Val, and wherein this Xaa is present where the Xaa in the third amino acid position is absent
  MISC_FEATURE (3)..(3): Xaa is present or absent, and if present, Xaa is Asn, Arg, Asp, Cys, Gln, Glu, Gly, Ser, Thr, or Tyr, and wherein this Xaa is present where the Xaa in the second amino acid position is absent
  MISC_FEATURE (5)..(5): Xaa is Arg, His, or Lys
  MISC_FEATURE (6)..(6): Xaa is Arg, Asp, Glu, His, Lys, Phe, Trp, or Tyr
  MISC_FEATURE (7)..(7): Xaa is Asn, Cys, Gln, Gly, Ser, Thr, Tyr, or Val
  MISC_FEATURE (8)..(8): Xaa is present or absent, and if present, Xaa is Asn, Gly, His, Ile, Thr, or Val
  MISC_FEATURE (10)..(10): Xaa is present or absent, and if present, Xaa is Ala, Asn, Ile, Leu, Met, Phe, Pro, Thr, Trp, or Val
XXXCXXXXTXG

SEQ ID NO: 2 - protein, 9 aa, Artificial Sequence
  Peptide based on human Netrin-1 sequence
  MISC_FEATURE (1)..(1): Wherein diethylene glycol (mini-PEG) is linked thereto
CDCRHNTAG

SEQ ID NO: 3 - protein, 10 aa, Artificial Sequence
  Peptide based on human Netrin-1 sequence
  MISC_FEATURE (1)..(1): Wherein diethylene glycol (mini-PEG) is linked thereto
CLNCRHNTAG

SEQ ID NO: 4 - protein, 12 aa, Artificial Sequence
  Peptide based on human Netrin-1 sequence
  MISC_FEATURE (1)..(1): Wherein diethylene glycol (mini-PEG) is linked thereto
CPCKDGVTIGIT

SEQ ID NO: 5 - protein, 9 aa, Artificial Sequence
  Peptide based on human Netrin-1 sequence
SDCRHNTAG

SEQ ID NO: 6 - protein, 9 aa, Artificial Sequence
  Peptide based on human Netrin-1 sequence
TDCRHNTAG

SEQ ID NO: 7 - protein, 9 aa, Artificial Sequence
  Peptide based on human Netrin-1 sequence
  DISULFID (1)..(3)
CDCRHNTAG

SEQ ID NO: 8 - protein, 9 aa, Artificial Sequence
  Peptide based on human Netrin-1 sequence
  MISC_FEATURE (1)..(1): D-amino acid residue
  MISC_FEATURE (9)..(9): D-amino acid residue
CDCRHNTAG

SEQ ID NO: 9 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 10 - protein, 9 aa, Artificial Sequence
  Peptide based on human Netrin-1 sequence
CDCRHNTAG

SEQ ID NO: 11 - protein, 10 aa, Artificial Sequence
  Peptide based on human Netrin-1 sequence
CLNCRHNTAG

SEQ ID NO: 12 - protein, 11 aa, Artificial Sequence
  Peptide based on human Netrin-1 sequence
CPCKDGVTGIT

SEQ ID NO: 13 - protein, 54 aa, Artificial Sequence
  Peptide based on human Netrin-1 sequence
CKCNGHAARCVRDRDDSLVCDCRHNTAGPECDRCKPFHYDRPWQRATAREANEC

SEQ ID NO: 14 - protein, 61 aa, Artificial Sequence
  Peptide based on human Netrin-1 sequence
CNCNLHARRCRFNMELYKLSGRKSGGVCLNCRHNTAGRHCHYCKEGYYRDMGKPITHRKAC

SEQ ID NO: 15 - protein, 48 aa, Artificial Sequence
  Peptide based on human Netrin-1 sequence
CDCHPVGAAGKTCNQTTGQCPCKDGVTGITCNRCAKGYQQSRSPIAPC

SEQ ID NO: 16 - protein, 36 aa, Artificial Sequence
  Peptide based on human Netrin-1 sequence
NLHARRCRFNMELYKLSGRKSGGVCLNCRHNTAGRH

SEQ ID NO: 17 - protein, 27 aa, Artificial Sequence
  Peptide based on human Netrin-1 sequence
HPVGAAGKTCNQTTGQCPCKDGVTGIT